(19) United States
(12) Patent Application Publication
(10) Pub. No.: US 2025/0082891 A1
(43) Pub. Date: Mar. 13, 2025

(54) PROTECTION OF USER SLEEP THROUGH EVALUATION OF A DISTURBANCE EVENT AGAINST A SLEEP PRIORITY STATE AND/OR A SLEEP PRIORITY PERIOD

(71) Applicant: Stealth Labs Inc., Brookline, MA (US)

(72) Inventors: YADID AYZENBERG, Brookline, MA (US); AMIR LAZAROVICH, Apex, NC (US)

(21) Appl. No.: 18/244,365

(22) Filed: Sep. 11, 2023

(51) Int. Cl. A61M 21/02; G06F 3/01; G06F 40/20; G10K 11/178; H04R 1/10

(52) U.S. Cl. CPC A61M 21/02; G06F 3/017; G06F 40/20; G10K 11/17823; G10K 11/17827; G10K 11/17873; G10K 11/17885; H04R 1/1041; H04R 1/1083; A61M 2021/0027; A61M 2205/3303; A61M 2205/3375; A61M 2205/3584; A61M 2205/502; A61M 2205/52; A61M 2230/18; G10K 2210/1081; G10K 2210/3027; G16H 50/70; H04R 2460/01

(57) ABSTRACT

Disclosed is a method, a device, and/or a system of protection of user sleep through evaluation of a disturbance event, such as an environmental sound or a communication, against a sleep priority state and/or a sleep priority period. In one embodiment, a device for enhancing sleep of a user, which may be embodied in an earbud case, may include an indicator identification subroutine that extracts an indicator data from a physiological data received from one or more sensors of an earphone, such as an earbud. A cognitive state evaluation routine determines a cognitive state of the user, for example a sleep state, based on a physiological feature. A communication agent receives a communication notification, for example from a communication device. A sleep protection routine determines that the user is in the sleep state and denies passthrough of the communication notification to protect the sleep of the user.

### FIELD OF TECHNOLOGY

This disclosure relates generally to data processing devices and, more particularly, to a method, a device, and/or a system of protection of user sleep through evaluation of a disturbance event against a sleep priority state and/or a sleep priority period.

### BACKGROUND

Sleep is an important part of health and wellbeing. However, it can be increasingly difficult for some people to achieve consistent, comfortable, and/or restful periods of sleep. This can especially be true because of the diversity of sleep environments, personal schedules, genetic tendencies, health conditions, and other individualized and/or group needs involving sleep.

As one example, certain users sleep in environments subject to random or consistent disruptions, such as communication devices (e.g., phones, apps) and/or environmental sounds (e.g., children, external street traffic, etc.). As another example, certain groups of users have related or interdependent sleep patterns (e.g., a couple who is married, military personnel in barracks, astronauts working on a space station). As another example, some users benefit greatly from naps, others need only a single rapid eye movement (REM) cycle per day, while still others require an uninterrupted seven hours of sleep each night.

One strategy to achieve sleep is for a user to block out sound, for example with ear plugs or noise canceling headphones. However, one aspect of sleep comfort and quality can be the state of mind of the user that they will not miss an event that, while disruptive, may be important. Worrying about such an event may prevent the user from relaxing and achieving a restful sleep. The user may have to choose between allowing all disturbances as to not miss an important disturbance, or tuning all disturbances out and risk missing an important event. Either choice may inhibit the cause cognitive distress and therefore sleep.

Good and/or sufficient sleep can also be seen as having economic value, both to individuals (e.g., for self-productivity and health), and also from the perspective of organizations that employ personnel, especially for jobs requiring a rested, alert, and creative workforce. There is a continuing need for new technologies that assist users in achieving consistent, comfortable, restful, and/or quality sleep, especially technologies that may be adaptable to the sleep needs, environments, and/or circumstances of individuals and/or groups.

### SUMMARY

Disclosed are a method, a device, and/or a system of protection of user sleep through evaluation of a disturbance event against a sleep priority state and/or a sleep priority period.

In one embodiment, a device for enhancing sleep of a user includes a housing, a processor, a network interface controller, and a memory. The network interface controller is configured to communicatively couple the device to an earphone of the user and a communication device and/or server. The memory includes an indicator identification subroutine, a feature extraction routine, a cognitive state evaluation routine, a communications agent, and a sleep protection routine.

The indicator identification subroutine includes computer readable instructions that when executed extract an indicator data from a physiological data received from one or more sensors of the earphone of the user. The indicator data indicates a respiration of the user, a heartbeat of the user, and/or a macro movement of the user. A feature extraction routine includes computer readable instructions that when executed determine a physiological feature of the user including a respiration rate of the user, a heart rate of the user, a heart rate variability of the user, and/or a macro movement period of the user, and assemble a feature data including the physiological feature.

The cognitive state evaluation routine includes computer readable instructions that when executed determine a cognitive state of the user based on the physiological feature, and record the cognitive state in association with a sleep session data. The communications agent includes computer readable instructions that when executed receive a communication notification that is a call notification, a message notification, and/or an app notification.

The sleep protection routine includes computer readable instructions that when executed query the sleep session data, determine that the user is in a sleep state and/or a pre-sleep state, and deny passthrough of the communication notification to a speaker of the earphone to protect the sleep of the user.

The device may include a sleep structuring engine that includes computer readable instructions that when executed initiate a sleep priority map and apply a first sleep priority value to a first period of the sleep priority map to designate a high priority period. The first period may be a user defined period and/or a REM period. The sleep structuring engine may include computer readable instructions that when executed apply a second sleep priority value to a second period of the sleep priority map to designate a low priority period. The second period may be a different user defined time period and/or an NREM period.

The device may include a communication classification engine that includes computer readable instructions that when executed classify the communication notification as a low priority communication. The sleep protection engine may further include computer readable instructions that when executed query the sleep session data, determine that the user is in the high priority period, and prevent a perceptible manifestation of the communication notification to protect a high priority sleep period of the user.

The device may include an audio collection agent that includes computer readable instructions that when executed receive an environmental sound from the environment of the user collected on a microphone of the device, a microphone of the communication device, and/or a microphone of the earphone, and then generate an audio data from the environmental sound. The device may also include a sound classification engine that includes computer readable instructions that when executed compare the audio data to an sound signature in an sound signature library, where the sound signature may be collected and added to the sound signature library by the user, and then classify the audio data as a high priority sound.

The sleep protection engine may further include computer readable instructions that when executed determine a sound priority level of the audio data exceeds a sleep priority level, and bypass an active noise canceling of the earphone to allow passthrough of the environmental sound and/or recreate the environmental sound on the speaker of the earphone. One or more of these capabilities may assist in initiating the sleep of the user by reassuring the user that important sounds will interrupt a high priority sleep state.

The user may be a first user and the earphone may be a first earphone of the first user and the device configured for communicative coupling to a second earphone of a second user simultaneously with the first earphone of the first user. The memory may further include a sleep grouping subroutine including computer readable instructions that when executed associate a device ID of the first earphone with a user profile of the first user, associate a device ID of a second earphone with a user profile of a second user, and designate a sleep group profile including the user profile of the first user and the user profile of the second user. The device may further include an event designation subroutine including computer readable instructions that when executed define an event data, and assign the event data to the user profile of the second user.

A disturbance classification engine may be included on the memory, which may include computer readable instructions that when executed receive a disturbance event, and determine the disturbance event is defined in the event data. The device may also include a group allocation subroutine including computer readable instructions that when executed query the sleep group profile, the user profile of the first user, and/or the user profile of the second user to determine the second user is associated with the event data that is associated with the second user profile. The sleep protection engine may further include computer readable instructions that when executed generate an audio indicator of the event data on the earphone of the second user to protect the sleep state of the first user.

The sleep structuring engine may further include computer readable instructions that when executed receive a sleep priority value designating a sleep priority level of a sleep period and/or a sleep cycle of the user. The communication classification engine may further including computer readable instructions that when executed extract a text data from a communication associated with the communication notification, query an artificial neural network, and determine a communication priority level of the communication based on the text data. The sleep protection engine may further include computer readable instructions that when executed determine the communication priority level of the communication exceeds the sleep priority level, and transmit a sound data associated with the communication notification to the speaker of the earphone, to assist in initiating the sleep of the user by reassuring the user that only important communications will interrupt the sleep period and/or the sleep cycle of the user.

The device may further include a display, and the memory may further include a sleep proportion subroutine. The sleep proportion subroutine may include computer readable instructions that when executed: (i) reference a target sleep duration value that is a default duration value and/or a custom duration value of the user, (ii) determine an actual sleep duration value based on the sleep session data, and (iii) calculate a proportion that is the actual sleep duration value relative to the target sleep duration value. A sleep sufficiency subroutine may include computer readable instructions that when executed generate a first graphical representation of the proportion of the actual sleep duration value relative to the target sleep duration value through a first graphical element representing an extent of the actual sleep duration value and/or a second graphical element representing the proportion. The first graphical representation may decrease a cognitive load of the user in interpreting a sufficiency of the sleep. The first graphical element represents a container and the second graphical element represents a filled portion of the container.

The device may also include a gesture agent that includes computer readable instructions that when executed receive a sleep level query generated by a gesture input on a touch interface of the earphone. The sleep sufficiency subroutine may include computer readable instructions that when generated (a) display a clock on the display, (b) generate a second graphical representation of the proportion of the actual sleep duration value relative to the target sleep duration value including a third graphical element representing the proportion with a color, the second graphical representation to decrease the cognitive load of the user in interpreting the sufficiency of the sleep, (c) generate a sound representation of the proportion of the actual sleep duration value relative to the target sleep duration value that includes (i) a tone representing the proportion and/or (ii) a word describing the proportion, the sound representation played on the speaker of the earphone.

The physiological data and/or the indicator data received from the earphone at a rate of between 20 Hz and 700 Hz. The physiological data, the indicator data, and/or audio data may be received on a low energy protocol, and the coordination hub may be pairable with a device generating the communication notification is recognizable by the device as an earphone device through a pairing communication protocol.

In another embodiment, a method for protecting sleep of a user includes receiving a physiological data that includes a gyroscope data, an accelerometer data, a temperature data of the user, and/or a sound of the user, and optionally receiving an ambient temperature of an environment of the user. The physiological data is received on one or more sensors of an earphone. The method extracts an indicator data that indicates a respiration of the user, a heartbeat of the user, and/or a macro movement of the user. The physiological data is transmitted to a coordination hub. A physiological feature of the user is determined, the physiological feature including a respiration rate of the user, a heart rate of the user, a heart rate variability, and/or a macro movement period of the user. The method assembles a feature data that includes the physiological feature, determines a cognitive state of the user based on the physiological feature, and records the cognitive state in association with a sleep session data.

A communication notification that a call notification, a message notification, and/or an app notification, is received. The method queries the sleep session data, determines that the user is a sleep state or a pre-sleep state, and denyies passthrough of the communication notification to a speaker of the earphone to protect the sleep of the user.

In another embodiment, a system for managing sleep of a user. The system includes an earphone, a coordination hub, and a network communicatively coupling the earphone to the coordination hub. The earphone includes a speaker of the earphone, one or more sensors configured to gather a physiological data of a user, and a network interface controller of the earphone.

The communication hub may include a housing of the communication hub, a processor of the communication hub, a memory of the communication hub, a network interface controller of the communication hub, and a memory of the communication hub.

The memory of the communication hub includes an indicator identification subroutine that includes computer readable instructions that when executed extract an indicator data from a physiological data received from one or more sensors of the earphone of the user, the indicator data indicating a respiration of the user, a heartbeat of the user, and/or a macro movement of the user. The physiological data may be received from the earphone on a low energy protocol.

The memory of the coordination hub further includes a feature extraction routine that includes computer readable instructions that when executed determine a physiological feature of the user that includes a respiration rate of the user, a heart rate of the user, a heart rate variability of the user, and/or a macro movement period of the user, and assemble a feature data that includes the physiological feature.

The memory also includes a cognitive state evaluation routine, a communications agent, and/or a sleep protection routine. The cognitive state evaluation routine includes computer readable instructions that when executed determine a cognitive state of the user based on the physiological feature, and record the cognitive state in association with a sleep session data. The communications agent includes computer readable instructions that when executed receive a communication notification that is a call notification, a message notification, and/or an app notification. The sleep protection routine includes computer readable instructions that when executed query the sleep session data, determine that the user is a sleep state and/or a pre-sleep state, and deny passthrough of the communication notification to a speaker of the earphone to protect the sleep of the user. The network interface controller of the coordination hub is configured to communicatively couple to the earphone.

### BRIEF DESCRIPTION OF THE DRAWINGS

The embodiments of this disclosure are illustrated by way of example and not limitation in the figures of the accompanying drawings, in which like references indicate similar elements and in which:

FIG. 1 illustrates a sleep protection network including a user within a sleep environment utilizing earphones, a coordination hub detecting and evaluating a cognitive state of the user and/or managing communication of disturbance events to protect the sleep pf the user (such as communications and/or environmental sounds), a communication device that may be associated with the user, and a network coupling the earphones, the coordination hub, and/or the communication device, according to one or more embodiments.

FIG. 2 illustrates the coordination hub of FIG. 1, according to one or more embodiments.

FIG. 3 illustrates one or more earphones of FIG. 1, according to one or more embodiments.

FIG. 4 illustrates the communication device of FIG. 1, according to one or more embodiments.

FIG. 5A illustrates a first sleep map usable to create a sleep program adaptable to the individual and/or group needs of one or more users and against which the priority of disturbance events can be measured, according to one or more embodiments.

FIG. 5B illustrates a second example of a sleep map, according to one or more embodiments.

FIG. 5C illustrates a third example of a sleep map, according to one or more embodiments.

FIG. 5D illustrates a fourth example of a sleep map, according to one or more embodiments.

FIG. 5E illustrates a fifth example of a sleep map, according to one or more embodiments.

FIG. 6 illustrates a group sleep network in which two or more users may have disturbance events allocated to one or more members of a sleep group to maximize sleep of the sleep group and/or prevent groupwide disruption, according to one or more embodiments.

FIG. 7 illustrates a sleep protection process flow, according to one or more embodiments.

FIG. 8 illustrates a physiological and/or environmental data process flow, according to one or more embodiments.

FIG. 9 illustrates a cognitive state determination process flow, according to one or more embodiments.

FIG. 10 illustrates a sleep map assembly process flow, according to one or more embodiments.

FIG. 11 illustrates a communication protection process flow, according to one or more embodiments.

FIG. 12 illustrates an environmental sound protection process flow, according to one or more embodiments.

FIG. 13 illustrates a sleep group assembly process flow, according to one or more embodiments.

FIG. 14 illustrates a group disturbance event protection process flow, according to one or more embodiments.

FIG. 15 illustrates a low cognitive load sleep query process flow, according to one or more embodiments.

FIG. 16 illustrates an example of the sleep protection network utilizing a set of earbuds, an earbuds case also implementing the coordination hub, and a smartphone implementing the communication device, according to one or more embodiments.

Other features of the present embodiments will be apparent from the accompanying drawings and from the detailed description that follows.

### DETAILED DESCRIPTION

Disclosed are a method, a device, and/or system of protection of user sleep through evaluation of a disturbance event against a sleep priority state and/or a sleep priority period. Although the present embodiments have been described with reference to specific example embodiments, it will be evident that various modifications and changes may be made to these embodiments without departing from the broader spirit and scope of the various embodiments.

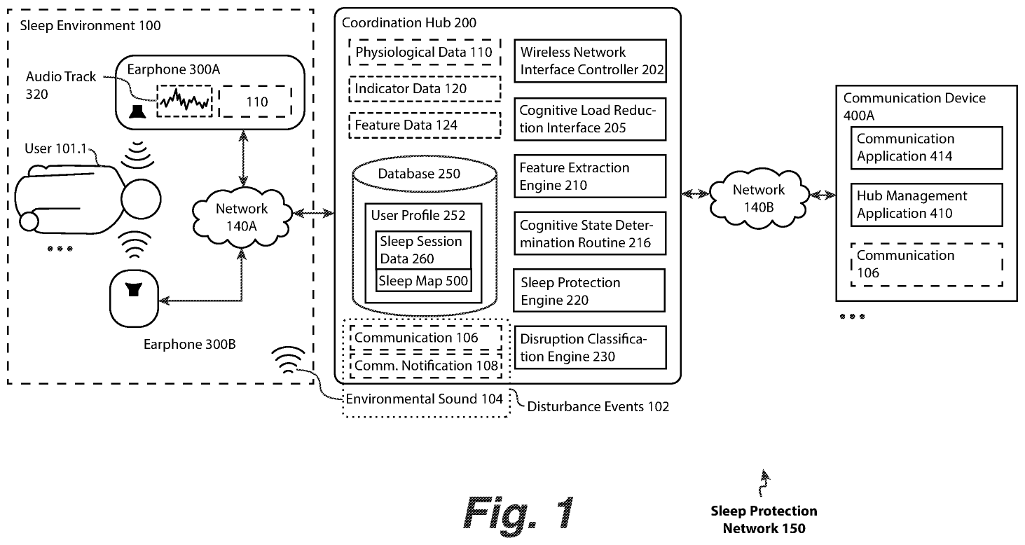

FIG. 1 illustrates a sleep protection network 150, according to one or more embodiments. The sleep protection network 150 may include one or more earphones 300 used by one or more users 101, a coordination hub 200, one or more communications devices 400, and a communication network referred to as the network 140 (e.g., a wireless network, a local area network, a WiFi network, the internet).

In one or more embodiments and the embodiment of FIG. 1, a user 101 may desire to sleep within a sleep environment 100. In one or more embodiments, the sleep environment 100 is the area around the user 101 that may generate audible noise and/or devices that may communicate with the user 101. The sleep environment 100 may be, for example, the bedroom of the user 101 and audible area outside of the home of the user 101, a public space such as an airport or train car, a group sleeping area such as a barracks with bunkbeds, etc. The user 101 may utilizes earphones 300 to provide noise canceling, masking sounds, noise reduction, and/or as general earplugs. The earphones 300 may be of any style, including for example earbuds, over-ear headphones, on-ear earphones, in-ear earphones, clip-on earphones, hearing aids, etc. In one or more embodiments, a pair of earphones 300, shown as the earphone 300A and the earphone 300B, may be wirelessly communicatively coupled through the network 140 to the coordination hub 200 and/or the communication device 400. However, as will be shown and described throughout the present embodiments, the earphones 300 may be communicatively coupled to the coordination hub 200 through a first network 140A (e.g., a Bluetooth® network), and the coordination hub 200 may in turn be communicatively coupled to the communication device 400 through a second network 140B (e.g., a WiFi network, the Internet).

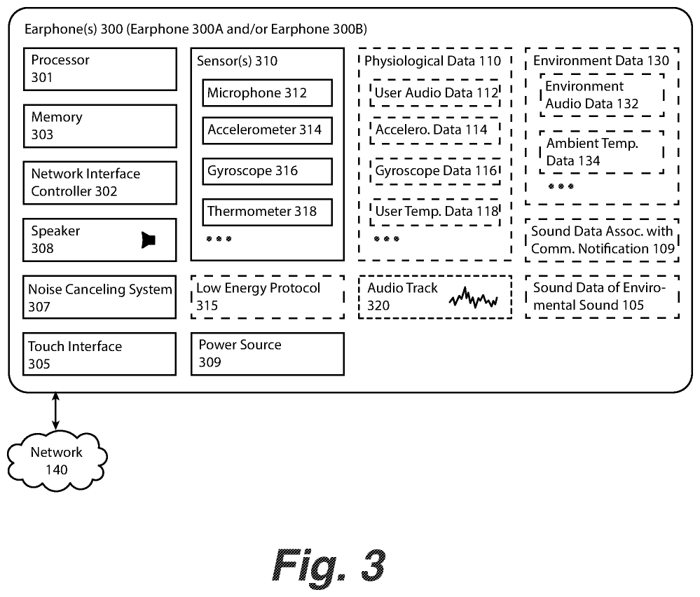

One or more sensors within the earphones 300 (e.g., the sensors 310 of FIG. 3) and/or sensors of the coordination hub 200 may be utilized to generate physiological data 110 and/or environmental data of the sleep environment 100, according to one or more embodiments. Physiological indicators may be extracted from the physiological data 110, for example indicators of a breath and/or respiration event of the user 101, a heartbeat of the user 101, and/or a macro movement of the user 101. For example, a microphone may be able to gather data of a sound indicating a respiration event of the user 101 and/or the heartbeat of the user 101, especially where the microphone is configured within the earphones 300 to gather sound from an inner-ear (e.g., blood rushing through the ear of the user 101).

Following generation of an indicator data 120, a feature extraction process such as can be applied by a feature extraction engine 210 may identify and extract physiological data, and/or define and store a feature data 124. Continuing the present example, the feature data 124 may include a respiration rate of the user 101, a heart rate of the user 101, a respiration rate variability (which may also be referred to as breath rate variability (BRV)), a heart rate variability (HRV) of the user 101, respiration trends and/or rate signatures, heart rate trends and/or rate signatures, and a macro movement period of the user 101 (e.g., the user 101 rolling over in bed, the user nodding off and jolting awake, the user 101 tossing and turning in bed). As just one example, a respiration trend and/or rate signature may describe a decrease of respiration rate followed by a rapid increase and/or spike in respiration rate, which may indicate the user 101 is entering a sleep state, as may be known in the art.

The feature data 124 may be utilized to determine a cognitive state of the user 101, for example an awake state, a pre-sleep state, a sleep state, a REM sleep state (referred to herein as the REM state), and/or a non-rapid eye movement state (e.g., an NREM sleep state, referred to herein as the NREM state). A cognitive state determination routine 216 may determine the cognitive state based one the feature data 124. The cognitive state, the feature data 124, the indicator data 120, and/or the physiological data 110 may be stored in a sleep session data 260, which may be optionally associated with a user profile 252 that may be associated with the user 101, the earphones 300, and/or the communication device 400. The sleep session data 260 may be queried to determine the extent of the sleep of the user 101, a present sleep state, an anticipated or forecast sleep state, and/or other data. In one or more embodiments, it will be noted that additional sensors may contribute to the determination of cognitive state of the user 101. For example, brainwave sensors or other sensors may be utilized to assist in determining cognitive state of the user 101. The sleep session data 260 may be periodically updated throughout the sleep of the user, for example every few seconds, minute, or ten-minute increment.

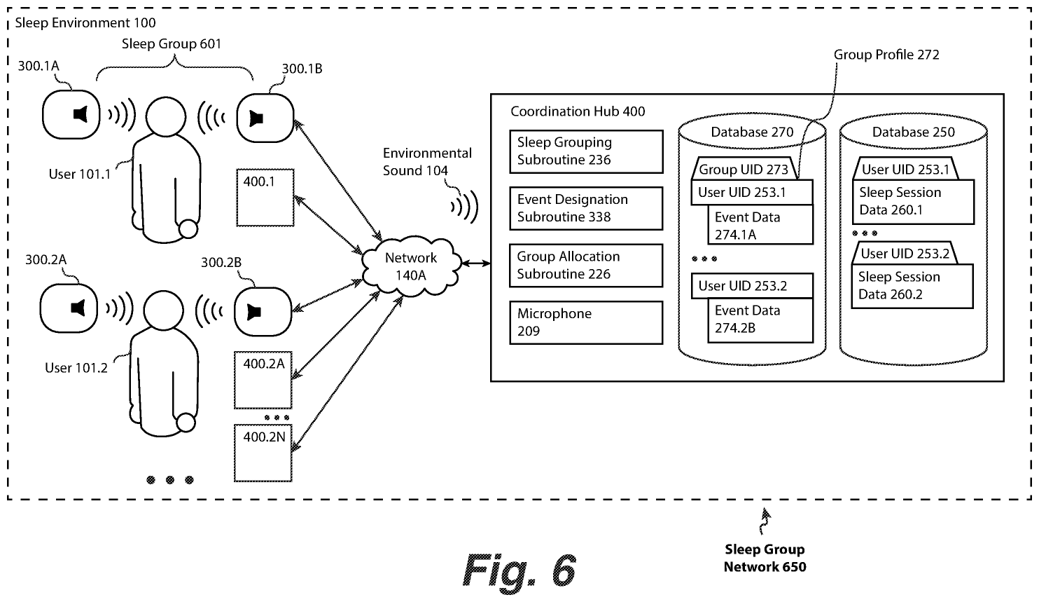

As further shown and described in conjunction with the embodiment of FIG. 6, it will be noted that user profiles 252 may be especially useful when more than one user 101 utilizes the same instance of the coordination hub 200. In one or more embodiments, it will be understood that one of the present advantages is that multiple instances of the user 101 may utilize the same instance of the coordination hub 200. Multiple instances of the coordination hub 200 (e.g., a coordination hub 200A, a coordination hub 200B, etc.) may be networked through the network 140, including to form a mesh network, according to one or more embodiments.

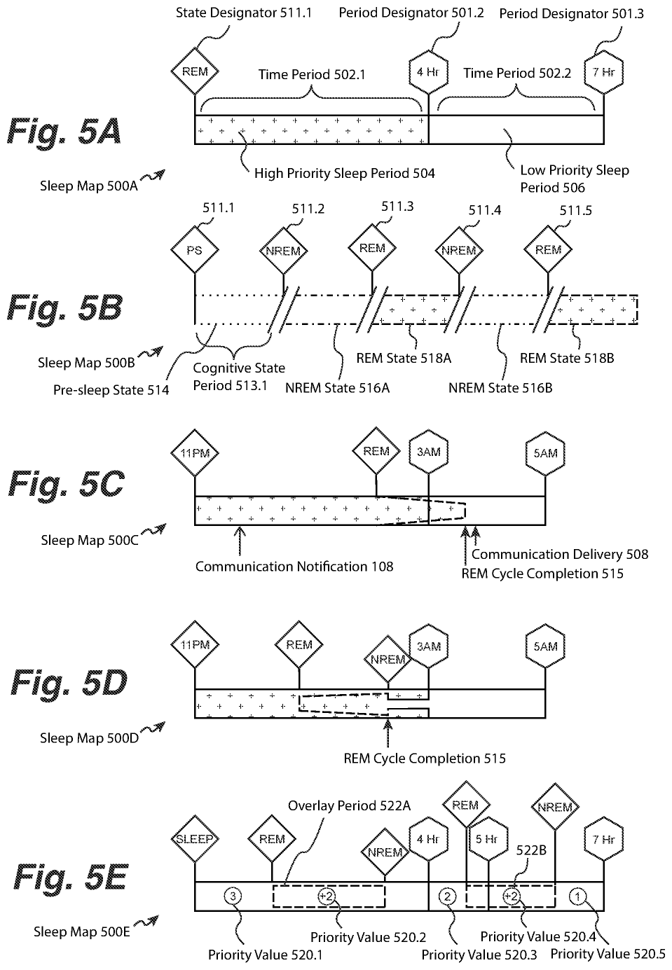

The coordination hub 200 may include default, predefined, and/or custom rules related to the prioritized sleep of the user 101. In one or more embodiments, and as further shown and described in conjunction with the embodiment of FIG. 5, a sleep map 500 may be generated which specifies time periods, cognitive states, and/or priority values for sleep related periods and/or events. In a straightforward example, a sleep map 500 may specify two sleep periods each lasting four hours, where the first period is a high priority sleep period, the second is a low priority sleep period, and where the first sleep period initiates upon the user 101 falling asleep (as may be determined from the cognitive state in the sleep session data 260). Additional examples of sleep maps 500, including more complex examples, are shown and described in conjunction with the embodiment of FIG. 6.

The user 101 may receive and/or may be subjected to one or more disturbance events. The disturbance event may be referred to as the disturbance event 102. A disturbance event 102 may include an environmental sound 104 (e.g., a car alarm, a siren, a baby crying, a loud neighbor, a howling wind, a phone ring, a barking dog, a crowing rooster, etc.) and/or a communication 106 (e.g., a phone call, an app message, a text message, an alert from an industrial equipment controller, etc.). Without earphones 300, the user 101 may be subjected to the disturbance event 102 which would possibly or probably wake the user 101 and/or disrupt a cognitive state (e.g., pre-sleep state to an awake state, the REM state to NREM state, etc.). When utilizing the earphones 300, the user 101 may reduce the probability of awakening and/or changing cognitive state. However, the user 101 may be concerned that some of the disturbance events 102 should, in fact, justifiably disturb the sleep of the user 101 (e.g., an infant of the user 101 is crying, a smoke alarm or fire alarm has been triggered, an urgent communication 106 is being received, etc.). The disturbance event 102 may include a communication 106 and/or an environmental sound 104.

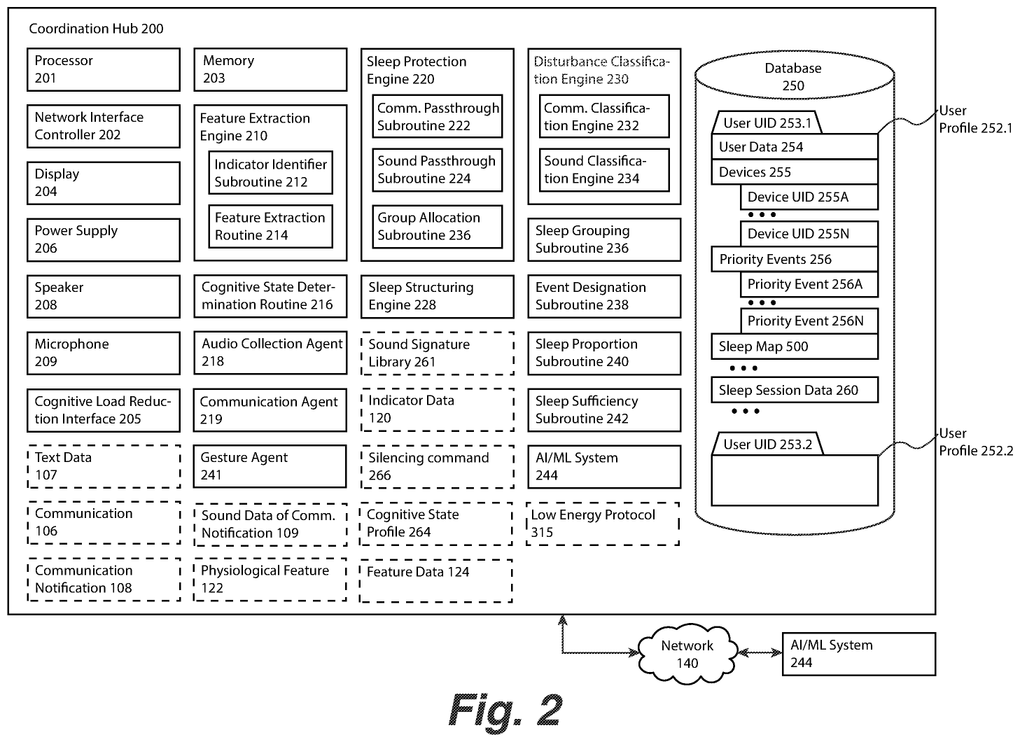

In one or more embodiments, the coordination hub 200 may include a sleep protection engine 220. The sleep protection engine 220 may be configured to protect the sleep of the user 101 by parsing incoming disturbance events 102. In one or more embodiments, the sleep protection engine 220 may query a disturbance classification engine 230 (e.g., as shown and further described in FIG. 2) that may classify and/or apply a priority value to a disturbance event 102. The sleep protection engine 220 may assess a priority of the disturbance event 102 relative to the sleep of the user 101, including referencing the sleep session data 260 and/or the sleep map 500 associated with the user 101. For example, it may be determined that the disturbance event 102 is a low-priority sound (e.g., a barking dog) and prevent passthrough of the sound past a noise canceling capability of the earphone 300. In another example, the disturbance event 102 that is a communication 106 from a business colleague may be of medium priority, but the user 101 may be in a REM state, where the communication notification 108 of the communication 106 is retained and communicated only after the user 101 transitions out of the REM state (e.g., enters an NREM state, wakes up).

The coordination hub 200 may be communicatively coupled to one or more instances of the communication device 400. The communication device 400 may be, for example, a smartphone, a mobile phone, a pager, a wearable device, a smartwatch, a tablet device, a laptop computer, a desktop computer, a server computer, etc. In one or more embodiments, the communication device 400 may also be a communication device integrated in and/or associated with an industrial control system and/or a vehicle (e.g., a self-driving car, a commercial aircraft). The communication device 400 may include a communication application 414 (e.g., a social media application, a workplace chat application, a video conferencing application, etc.). The communication device 400 may also include a hub management application 410 that may be utilized to manage the coordination hub 200, its configuration, and/or its settings. For example, in one or more embodiments the coordination hub 200 may lack a display and/or speaker, and the communication device 400 (such as a mobile device) may be used to connect and control and/or manage the coordination hub 200, and its data, software, settings, firmware, and/or other aspects.

In one or more embodiments, the coordination hub 200 may include a cognitive load reduction interface 205. The cognitive load reduction interface 205 may be utilized to communicate information related to the sleep of the user 101 while lowering the probability of changing the cognitive state of the user 101 (e.g., a pre-sleep state to an awake state) and/or awakening a different user 101 in a sleep group 601. In one or more embodiments, the cognitive load reduction interface 205 may include a visual and/or audible communication of a proportion of the sleep and/or proportion of high priority sleep the user 101 has achieved, as may be measured against either a default value, a custom value, and/or the sleep map 500 of the user 101. As just one example, the cognitive load reduction interface 205 may visually communicate, for example in the analogy of a filling battery graphical icon, the amount of REM sleep the user 101 has achieved (e.g., if the user achieved one of two desired REM sleep periods, the battery graphical icon may be displayed as half-full). As will be later shown and described, additional data also may be communicated at low cognitive load. As a result of the cognitive load reduction interface 205, the user 101 may be able to quickly (and at a lower probability of becoming wide awake) determine the sufficiency of their sleep, including relative to custom-defined and/or individualized sleep priorities.

As a result of the sleep protection network 150 and/or its individual components, one or more users 101 may be able to achieve more comfortable, relaxing, restful sleeps. In one or more embodiments, it will be recognized that an advantage of the sleep protection network 150 or portion thereof is to assist the user 101 in sleeping by providing piece of mind that only important disruptions will disrupt the sleep. In one or more embodiments, it will be recognized that an advantage of the sleep protection network 150 or portion thereof is to assist the user 101 in sleeping by providing piece of mind that a very important or urgent disruption will disrupt the sleep, lowing the probability the user 101 will sleep through the event. In one or more embodiments, it will be recognized that an advantage of the sleep protection network 150 or portion thereof is to assist the user 101 in sleeping by providing piece of mind that novel events not otherwise anticipated by the user 101 will wake the user 101.

In one or more embodiments, it will be recognized that an advantage of the sleep protection network 150 or portion thereof is to help protect the sleep of the user 101 by ensuring period of high value sleep, high importance sleep to the user, and/or high priority sleep periods designated by the user 101 are protected. In one or more embodiments, it will be recognized that an advantage of the sleep protection network 150 or portion thereof is to maximize the sleep quality and comfort of a group (e.g., the sleep group 601) by selectively disrupting the sleep of only one or a select subset users 101 within the group, as further shown and described in conjunction with the embodiment of FIG. 6. In one or more embodiments, it will be recognized that an advantage of the sleep protection network 150 or portion thereof is to assist the user 101 in falling back to sleep by providing low cognitive load information about sleep sufficiency and duration. Each of the components of the sleep protection network 150 will now be further described.

FIG. 2 illustrates the coordination hub 200, according to one or more embodiments. The coordination hub 200 may comprise a processor 201 (e.g., a computer processor, a microcontroller, and/or other computer instruction processing circuits), a memory 203 (e.g., a solid state memory, RAM, and/or other computer readable memory), and a network interface controller 202, for example a wireless network interface controller configured to communicate through one or more wireless protocols (e.g., Bluetooth®, low-energy Bluetooth, WiFi, cellular protocls, LTE, 5G, radio protocols, etc.). The coordination hub 200 may include a housing, and may take on various industrial form factors. In one or more embodiments, the coordination hub 200 may be a case for charging the earphones 300 in addition to one or more other functions described throughout the present embodiments. The coordination hub 200 may further include a display 204 (e.g., one or more indicator lights or LEDs, an LED display screen, an LCD display screen, an OLED display screen, etc.), one or more indicator lights (not shown in FIG. 2), a power supply 206 (e.g., an AC to DC converter, a battery), a speaker 208, and/or a microphone 209.

In one or more embodiments, the coordination hub 200 may include a feature extraction engine 210 that may be configured to determine and extract a physiological indicator from a physiological data 110 (e.g., as shown in FIG. 3) and/or may be configured to determine a physiological feature 122. For example, the feature extraction engine 210 may continually process data collected and transmitted by sensors 310 of the earphones 300 (and/or utilize additional data collected by the coordination hub 200). In one or more embodiments, the feature extraction engine 210 may include an indicator identifier subroutine 212 and/or a feature extraction routine 214. In one or more embodiments, the indicator identifier subroutine 212 may include computer readable instructions that when executed extract an indicator data 120 from a physiological data 110 (e.g., as further shown and described in conjunction with the embodiment of in FIG. 3) received from one or more sensors 310 of the earphone 300 of the user 101. The indicator data 120 may indicate, for example, a respiration of the user 101, a heartbeat of the user 101, and/or a macro movement of the user 101.

In one or more embodiments, the feature extraction routine 214 may include computer readable instructions that when executed determine a physiological feature 122 of the user 101 that may include, for example, a respiration rate of the user 101, a heart rate of the user 101, a heart rate variability of the user 101, and/or a macro movement period of the user 101. The feature extraction routine 214 may include computer readable instructions that when executed assemble a feature data 124 that includes the physiological feature 122. As one example, the earphones 300 may collect physiological data 110 and communicate the packages of data to the coordination hub 200 over the network 140 at a rate of between 20 Hz and 700 Hz, and in one or more preferred embodiments at a rate of 30 Hz to 40 Hz. The data package may include accelerometer data (e.g., x,y,z coordinates and/or axis coordinate data, as may be generated by a capacitive MEMS accelerometer, a piezoelectric accelerometer, a piezoresistive accelerometer, and/or another type of accelerometer), microphone audio data in one or more standard audio protocols, gyroscope data (e.g., specifying rotation values in each of the three or more axes), temperature data (e.g., temperature values in degrees Fahrenheit, Celsius, and/or in Kelvin. A resulting resolution for the indicator data 120 may be to identify physiological indicators at sub-second resolution. The features data 124 may, for example, identify physiological features over intervals, e.g., 1 second, 10 seconds, 30 seconds, and/or one minute.

In one or more embodiments, the coordination hub 200 may include a cognitive state determination routine 216 that may be configured to determine a cognitive state of the user 101 based on the physiological data 110, indicator data 120, physiological features 122, and/or feature data 124. The cognitive state of the user may include, for example, an awake state, a pre-sleep state, a sleep state, a REM state, a NREM state. Further fractionation of the cognitive state is also possible, for example certain phases of REM sleep, gradations of wakefulness and/or relaxation, etc. In one or more embodiments, cognitive state may be determined by assessment of one or more physiological features 122, alone or in combination.

In one or more embodiments, the cognitive state may be determined by the cognitive state determination routine 216 through one or more machine learning-based approaches that may utilize multiple inputs (e.g., respiration rate, respiration rate variability, respiration trend, heart rate, heart rate variability, previous sleep state, etc.) to determine a likelihood a user is in a particular cognitive state. The machine learning model may be supervised or unsupervised, as may be known in the art. In one or more embodiments, a machine learning model may be trained utilizing a supervised training dataset collected from one or more individuals monitored by one or more devices collecting the relevant inputs and observed for cognitive state (e.g., whether the user 101 responds to verbal prompts, whether the user 101 appears to an observer to be sleeping, an EEG signal collected from the user 101 through brainwave detection equipment, including as may be capable of detecting REM eye movements). We evaluate the user 101.1 state may be based on data collected over an epoch (e.g., 10 seconds, 30 seconds, 2 minutes). The user 101 may also be able to provide feedback to further refine and/or train one or more machine learning models and/or the cognitive state determination routine 216. For example, periodically the user may be asked by audio whether the user 101 is awake, and the user 101 may be able to provide a response if awake (e.g., through the touch interface 305, verbally through the microphone 312), or no response if asleep. The machine learning model may build and/or define one or more artificial neural networks and/or other models that may be applied by the cognitive state determination routine 216.

In one or more embodiments, rules-based systems may also be developed and applied by the cognitive state determination routine 216, whether along and/or in combination with machine learning. As an example, it may be determined that a heart rate above a reference value may indicate an awake state, whereas a heart rate of between a first value and a second value, accompanied by a respiration rate of between a third value and a fourth value may indicate a sleep state of the user 101.1. In one or more embodiments, the cognitive state determination routine 216 may include computer readable instructions that when executed determine a cognitive state of the user 101 based on one or more physiological features 122, and may optionally record the cognitive state in association with a sleep session data 260. For example, the sleep session data 260 may store the feature data 124 for a period of time (e.g., 10 seconds), and also store the determined cognitive state for that period of time (e.g., the user 101 was asleep during the period of time). The sleep session data 260 may be stored in a database 250 and/or in association with the user profile 252.

Data specifying the ruleset for determining cognitive state, and/or a “signature” for cognitive state based on physiological data, may be stored as a cognitive state profile 264. In one or more embodiments, custom data for user-specific and/or individualized cognitive state profiles 264 may be defined and stored in association with the user profile 252.

In one or more embodiments, the coordination hub 200 may include an audio collection agent 218. The audio collection agent 218 may be configured to collect audio from the user 101 and/or collect the environmental sound 104 from the sleep environment 100 of the user 101. The audio collection agent 218 may be collecting audio for analysis at all times, may collect audio upon detection of a sound over a certain decibel level, may collect sound data once the user enters a certain cognitive state, may collect audio upon determination of a certain type of sound or sound signature, etc. In one or more embodiments, the audio collection agent 218 may determine a direction and/or source of a sound, for example where the microphone 209 comprises one or more individual microphones of the coordination hub 200. This may help distinguish sounds of the user 101 from other sounds. As just one example, a first microphone 209A may be placed such that it is directed toward a sleeping area of the user 101, and a second microphone 209B may be placed such that it is directed away from the sleeping area of the user 101, where the microphone 209A may primarily be utilized to capture physiological data 110 (e.g., the user 101 breathing), whereas the microphone 209B may be primarily utilized to receive the environmental sound 104.

In one or more embodiment, the audio collection agent 218 may include computer readable instructions that when executed receive an environmental sound 104 from the environment (e.g., the sleep environment 100) of the user 101 collected on at least one of the microphone 209 of the coordination hub 200, a microphone 409 of the communication device 400, and/or a microphone 312 of the earphone 300. The audio collection agent 218 may include computer readable instructions that when executed generate an audio data from the environmental sound 104. The audio data may be classified, as further described below, for example by comparison against one or more sound signature, optionally stored within a sound signature library 261. Alternatively or in addition, the sound data may be classified by being submitted to an artificial intelligence and/or machine learning classification system trained with common sounds occurring within one or more sleep environments 100 and/or personalized training data collected in association with the user 101 and/or the user profile 252 of the user 101.

In one or more embodiments, the coordination hub 200 may include a communication agent 219 configured to receive a communication 106, a communication notification 108, and/or metadata thereof. The communication agent 219 may receive data from an API call and/or API notification from the communication device 400 and/or a different device or system, for example over the network 140. In one or more embodiments, the communication 106 may be a phone call, a voicemail, a text message, an SMS message, a pager message, an application message (e.g., from an “app” on a smartphone), etc. In one or more embodiments, the communication notification 108 may be a notification and/or metadata of the communication 106 used to alert, indicate, and/or notify a user 101 of the communication 106. For example, the communication notification 108 may be a push notification, an API call indicating a communication 106 is pending (e.g., a call is ringing) and/or is available for review (e.g., a voicemail or text message is available, including a possible preview thereof), etc. In one or more embodiments, the communications agent 219 may include computer readable instructions that when executed receive a communication notification 108 that a call notification, a message notification, and/or an app notification is pending. The communication notification 108 may be passed to one or more other sets of computer readable instructions, for example with a procedure call to the disturbance classification engine 230.

In one or more embodiments, the coordination hub 200 may include a disturbance classification engine 230 configured to receive data describing a disturbance event 102 and classify the disturbance event 102 into a type of disruption, a priority of disruption (e.g., ‘high’ or ‘low’, and/or a numerical value), an urgency of disruption, and/or other useful classifications. The disturbance event 102 may be, for example, an environmental sound 104 and/or a communication 106 or notification thereof. In one or more embodiments, the disturbance classification engine 230 may include a communication classification engine 232 and/or a sound classification engine 234. The communication classification engine 232 may be configured to receive a communication 106 and/or communication notification 108 of a communication 106, or metadata of either, and determine a priority, importance, and/or urgency of the communication 106. The sound classification engine 234 may be configured to receive an environmental sound 104 and determine a priority, importance, and/or urgency of the environmental sound 104.

Classification of a communication 106 and/or communication notification 108 may be carried out, for example, by comparison to a known list of important types of communications, senders of communications, complex rulesets (e.g., a call from a family member between 11 PM and 4 AM). Classification of a communication 106 and/or communication notification 108 may utilize additional metadata applied by the communication device 400, for example that a sender of the communication 106 is on a “favorites” list, on an “important” list, and/or within a certain work group (e.g., a software development operations (“dev ops”) team responsible for a server computer 24 hours a day, seven days a week).

An environmental sound 104 may be classified through general characteristics of an acoustical wave against a ruleset (e.g., any sound with predominant components over 5000 Hz, any sound greater than 60 decibels, and/or any loud, recuring, high-pitched sound with similar waveform, such as may be generated by a fire alarm, smoke alarm, and/or carbon monoxide alarm etc.). The environmental sound 104 may also be classified according to matching against a sound signature 262, in which the waveform of the environmental sound 104 may be compared to the sound signature 262 to determine a match within a confidence range. Multiple comparison processes, filters, and/or waveform analysis may be performed to match against the sound signature 262 as a “fingerprint” for a sound and/or type of sound, as may be known in the art. One or more sound signatures 262 may be stored in a sound signature library 261, according to one or more embodiments. The sound signature library 261 may be developed by a company, organization, and/or enterprise operating and/or selling the coordination hub 200, may be created and/or augmented by the user 101, and/or may be periodically updated with common sound signatures 262 over the network 140. For example, the sound signature library 261 may include sound signatures 262 for barking dogs, common construction equipment, distant sirens, honking, and other sounds that may be generally considered disruptive noise. The sound signatures 262 may also include sounds that, while disruptive, may be important, for example smoke alarms, carbon monoxide alarms, crying infants, etc.

Priorities (e.g., “high” or “low”) and/or priority values (e.g., “1” through “10”) may be assigned to each disturbance event 102 that may be able to be classified. For example, data associated with the sound signature 262 may include the priority value. Priority and/or priority value may also be contextual. For example, the user 101 may be able to select a travel mode, where sounds not ordinarily considered to be low-priority disruptions are treated as such (e.g., sounds of a plane taking off, etc.). In one or more embodiments, priority value may increase as the disturbance event continues (e.g., a baby crying for more than two minutes, or more than several times within 30 minutes).

In one or more embodiments, an artificial intelligence system and/or machine learning system (referred to as the AI/ML system 244) may be utilized in classifying disturbance events 102, and the disturbance classification engine 230 may general a procedure call to an AI/ML trained model for classification. According to one or more embodiments, the model also may be instantiated with custom data submitted by each user 101, for example to custom tailor what may be considered disadvantageous environmental noise and/or communications versus an important disruption. In one or more embodiments, feedback may be provided by the user 101 in realtime, for example by the user 101 providing input via a gestural interface of the earphones 300 or other control interface of the coordination hub 200. Such feedback may be used to assist in training the AI/ML system 244 for the particular user, and/or for the benefit of other users 101 that may receive updates from the coordination hub 200.

In one or more embodiments, the AI/ML system 244 may be able to assist in recognizing events that, while unanticipated or never before experienced by the user 101 nevertheless are urgent. Anonymized training sets drawn from data gathered from multiple coordination hubs 200 may assist in developing a generalizable model of disturbance events 102 with a high priority value (e.g., which have a high probability of being disturbance events 102 the user 101 would wish to be woken for). In one or more embodiments, it will be recognized that an advantage of the sleep protection network 150 or portion thereof, including the AI/ML system 244, is to assist the user 101 in sleeping by providing piece of mind that novel events not otherwise anticipated by the user 101 will wake the user 101.

In one or more embodiments, the communication classification engine 232 may include computer readable instructions that when executed classify the communication notification 108, for example as a low priority communication, a medium priority communication, or a high priority communication. In one or more embodiments, the sound classification engine 234 may include computer readable instructions that when executed compare the audio data to an sound signature 262 in a sound signature library 261, and then classify the audio data as a low priority sound, a medium priority sound, or a high priority sound. The sound signature 262 may have been collected and added to the sound signature library 261 by the user 101. For example, the user may have recorded their dog barking for categorization as “low priority”, and recording their infant crying for categorization as “high priority”.

In one or more embodiments, it may be useful to parse any text data 107 resulting from a communication 106 to determine communication priority. The text data 107 may be preexisting in the communication 106 (e.g., text of a text message), and/or may be generated from the communication 106 (e.g., voice-to-text applied to a voicemail message or even an environmental sound 104 of a person speaking near the user 101). The text data 107 may be useful as an input to the AI/ML system, including analysis by large language models (LLMs) trained to determine urgency and/or importance of text data related to a communication 106. For example, proper training of the model is likely to be able to distinguish a general advertisement or solicitation texted to the phone of the user 101, versus a family member informing the user 101 that another mutual family member has just been admitted to the hospital. In one or more embodiments, the communication classification engine 232 may include computer readable instructions that when executed extract a text data 107 from a communication 106 associated with a communication notification 108, query an AI/ML system 244 such as an artificial neural network (ANN) and/or large language model, and determine a communication priority level of the communication based on the text data 107 (e.g., as an output of the AI/ML system 244).

The coordination hub 200 may include a sleep protection engine 220 configured to receive disturbance events 102 that may have been classified (e.g., by the disturbance classification engine 230) and determine whether to permit or deny the disturbance event 102 from disturbing the cognitive state and/or the sleep state of the user 101. The disturbance may be permitted, for example, by passing the audio data generated from an environmental sound 104 to the speaker 308 of the earphone 300, by turning off active noise canceling system 307, and/or by allowing other signaling such as vibratory notification of the earphones 300. The sleep protection engine 220 may determine disturbance event 102 outcome by comparison to a ruleset (e.g., all low priority disturbance event 102 are denied, whereas all high priority disturbance events 102 are permitted), query of the user 101 sleep state (e.g., all low priority disturbance events 102 are denied if the user 101 is in a pre-sleep state or a sleep state, all medium priority disturbance events 102 are denied if the user 101 is in the REM sleep state, and all high priority disturbance events 102 are permitted).

In one or more embodiments, the sleep protection engine 220 may include a communication passthrough subroutine 222, a sound passthrough subroutine 224, and/or a group allocation subroutine 226 (the group allocation subroutine 226 is further shown and described in conjunction with the embodiment of FIG. 6). The communication passthrough subroutine 222 may be configured to determine whether a communication 106, and/or communication notification 108 is permitted to disturb the user 101, including possibly via passthrough to the speaker 308 of the user 101.

In one or more embodiments, the communication passthrough subroutine 222 may compare a communication priority level of the communication 106 and/or the communication notification 108 to the sleep priority level to determine which may take priority. In one or more embodiments, the communication passthrough subroutine 222 may include computer readable instructions that when executed query the sleep session data 260, determine that the user 101 is in a sleep state and/or a pre-sleep state, and deny passthrough of the communication notification 108 to a speaker 308 of the earphone 300 to protect the sleep of the user 101. Alternatively, or in addition, communication notifications 108 and/or communications 106 may be held and delivered according to certain rules, for example when the user 101 returns to a certain cognitive state (e.g., an awake state, an NREM sleep state, etc.).

In one or more embodiments, the communication passthrough subroutine 222 may include computer readable instructions that when executed query the sleep session data 260, determine that the user 101 is in a high priority period (e.g., a REM sleep state, and/or a high priority level as may be defined in the sleep map 500), and prevent a perceptible manifestation of the communication notification 108 to protect a high priority sleep period of the user 101. The perceptible manifestation may be sight, sound, and/or feeling (e.g., vibration).

In one or more embodiments, the communication passthrough subroutine 222 may include computer readable instructions that when executed determine if the communication priority level of the communication 106 exceeds the sleep priority level. The computer readable instructions, when executing, may then transmit a sound data associated with the communication notification 108 (e.g., a chime, a beep, a verbal description of the communication 106 from a synthesized or recorded voice) to the speaker 308 of the earphone 300. This may assist in initiating the sleep of the user 101 by reassuring the user 101 that only important communications 106 will interrupt the sleep period and/or the sleep cycle of the user 101.

The sleep protection engine 220 may also issue a silencing command 266 to the communication device 400 generating the communication 106 and/or communication notification 108. The silencing command may instruct the communication device 400, one or more applications thereon, and/or the operating system 406 to “silence” or otherwise turn off the communication 106 and/or communication notification 108, including rings, beeps, vibrations, or other perceptible manifestations that could disrupt the sleep of the user 101 if the communication device 400 is within the sleep environment 100.

In one or more embodiments, the sound passthrough subroutine 224 may include computer readable instructions that when executed compare a sound priority level of the audio data (e.g., of the environmental sound 104) to a sleep priority level to determine which may take priority. In one or more embodiments, the sound passthrough subroutine 224 may include computer readable instructions that when executed determine a sound priority level of the audio data (e.g., of the environmental sound 104) exceeds a sleep priority level, and (a) bypass an active noise canceling and/or masking (e.g., of the noise canceling system 307) of the earphone 300 to allow passthrough of the environmental sound 104, and/or (b) recreate the environmental sound 104 on the speaker 308 of the earphone 300. This may assist in initiating the sleep of the user 101 by reassuring the user 101 that important sounds (e.g., the environmental sound 104) will interrupt a sleep state, including optionally a high priority sleep state.

In one or more embodiments, the sleep protection engine 220 may reference a sleep map 500 and/or the sleep session data 260 when determining the priority of the sleep or other cognitive state of the user 101. In one or more embodiments, the sleep protection engine 220 may query the sleep map 500 to determine which portion or period of the sleep map 500 in which the user 101 may be currently “located”, as further shown and described in conjunction with the embodiment of FIG. 5A through FIG. 5E. In one or more other embodiments, the sleep protection engine 220 may query the sleep session data 260 to determine the present cognitive state of the user 101. In one or more other embodiments, the sleep protection engine 220 may query both the sleep session data 260 and the sleep map 500, which may be especially useful where the sleep map 500 is dependent upon, references, and/or includes reference to the cognitive state of the user 101, as further shown and described in conjunction with FIG. 5.

A sleep structuring engine 228 may be configured to define a sleep map 500 as a data structure within the memory 203 and/or other computer readable memory. The sleep map 500 may describe a temporal sequence of discrete, overlapping, and/or overlaid cognitive periods and cognitive states, each of which may be associated with one or more priority levels. The sleep map 500 may have a general structure (e.g., a “quick nap” profile), or may be highly customized. For example, the sleep map 500 could be used to help ensure an oceanic oil rig operator achieves multiple sleep objectives, including a full REM sleep cycle, is woken to check a critical controller and receive any high priority messages, and is able to then achieve another full REM cycle.

In one or more embodiments, a sleep structuring engine 228 includes computer readable instructions that when executed initiate a sleep priority map 500 (e.g., as a data structure inside a database), apply a first sleep priority value to a first period of the sleep priority map 500 to designate a high priority period, and apply a second sleep priority value to a second period of the sleep priority map 500 to designate a low priority period. For example, the first period may be (i) a user defined period and/or (ii) a REM period, and the second period may be (i) a different user defined time period and/or (ii) an NREM period. In one or more other embodiments, the sleep structuring engine 228 may include computer readable instructions that when executed receive a sleep priority value designating a sleep priority level of a sleep period and/or a sleep cycle of the user 101, and store the sleep priority value is association the sleep period and/or the sleep value of the user in the sleep map 500.

The coordination hub 200 may also include cognitive load reduction interface 205. In one or more embodiments, a common question that a user 101 may have while attempting to sleep and/or after waking may be (i) what time it is; (ii) how much the user 101 slept; (iii) and possibly more importantly, but often difficult to determine, how sufficiently rested the user 101 is. With respect to the current time, it is sometimes related to the second question (how much the user 101 has slept). In the case of a common bedside alarm clock, smartphone clock, or wristwatch, the user 101 may wish to look at the time for the purpose of determining how long the user 101 has slept. However, the assessment of the numerical values may pose a challenge in that (a) it may activate logical processes of the brain that increase wakefulness, (b) the user 101 may have to fumble with the device to activate or view the clock, and/or (c) the light from the clock, especially white or blue light, may enter the retina and cause a physiological response that increases wakefulness. The third question (how the rested the user 101 is) may be difficult to determine, as it requires both feeling present restfulness and possibly remembering the extent to which sleep or an awake state was expected over an intended sleep period. This may further result in cognitive load on the user 101 and further push the user 101 away from again achieving the sleep state. It may also cause disturbance of someone else sleeping nearby, such as a spouse or work colleague.

In one or more embodiments, the cognitive load reduction interface 205 may reduce cognitive load, and thereby encourage the sleep state. The cognitive load reduction interface 205 may comprise graphical and/or sound elements communicating sleep level and/or sleep sufficiency, including with relation to the sleep session data 260 and/or the sleep map 500.

In one or more embodiments, the cognitive load reduction interface 205 may include a graphical representation of sleep sufficiency that can be rapidly cognitively processed without resorting to mental math or character recognition. For example, the graphical representation may include an analogy of a “sleep battery”, either in color (e.g., red for depleted, yellow for partially “charged”, and green for fully “charged”) and/or in a filled proportion (e.g., an unfilled container for depleted, a partially filled container for partially “charged”, and a filled container for “charged”).

In one or more embodiments, the coordination hub 200 may include a sleep proportion subroutine 240 configured to calculate a proportion of the sleep of the user 101 relative to a target time period that may be specified as a default value (e.g., 7 hours), a target specified in the sleep map 500, and/or other user defined target. The sleep proportion subroutine 240 may include computer readable instructions that when executed reference a target sleep duration value that is a default duration value and/or a custom duration value of the user (as either may be defined within or without the sleep map 500), determine an actual sleep duration value based on the sleep session data 260, and calculate a proportion that is the actual sleep duration value relative to the target sleep duration value. The sleep proportion subroutine 240 may also base the proportion on sleep periods and/or sleep states of high priority, for example the total amount of REM sleep (e.g., time within a REM sleep state) that the user 101 has achieved relative to a target amount of REM sleep (e.g., 30% of the total sleep period within a sleep session, 4 hours within the sleep session, etc.).

In one or more embodiments, the coordination hub 200 may include a sleep sufficiency subroutine 242 configured to generate the representation of the sleep sufficiency through the cognitive load reduction interface 205. In one or more embodiments, the sleep sufficiency subroutine 242 includes computer readable instructions that when executed generate a graphical representation of the proportion of the actual sleep duration value relative to the target sleep duration value. The graphical representation may be generated through a first graphical element representing an extent of the actual sleep duration value (e.g., as queried from a default value, a custom value input by the user 101, a value inferred from the sleep patterns of the user 101 over several sleep sessions, and/or the sleep map 500) and a second graphical element representing the proportion. The first graphical element may be rendered such that it represents a container (e.g., an unfilled cup, an unfilled rectangle, another unfilled polygon) and the second graphical element may be rendered such that it represents a filled portion of the container (e.g., water in the cup, a partially or completely filled portion of the rectangle, a partially or completely filled portion of the polygon). Such graphical representation may decrease the cognitive load of the user 101 in interpreting the sufficiency of the sleep.

The sleep sufficiency subroutine 242 may include comprising computer readable instructions that when executed generate a graphical representation of the proportion of the actual sleep duration value relative to the target sleep duration value comprising a third graphical element representing the proportion with a color (e.g., a “heatmap” of color, a transition between two colors, discrete colors associated with bad/red, mediocre/yellow, and good/green, etc.). Such graphical representation may decrease the cognitive load of the user 101 in interpreting the sufficiency of the sleep. Other graphical representations may include a numerical percentage (which may still reduce cognitive load compared with the user evaluating a clock), a timer indicating the number of hours slept, and/or a “progress bar” along a linear, annular, arc, and/or circular path, etc.

In one or more embodiments, the cognitive load reduction interface 205 may include a sound representation of sleep sufficiency that can be rapidly cognitively processed without resorting to mental math, verbal word recognition, or visual processing by the eyes of the user 101. For example, one or more tones, chimes, or audio soundbites may indicate sleep proportion and/or sufficiency. In a more specific example, the sleep proportion may be fractionated into an eight-note scale, where each note may represent one-eighth of the proportion between empty and full (e.g., a low A note for 12.5% or less, a D note for 50%, a high A note one octave above the low A note for 100%). Other sounds or soundbites may be utilized as well (an owl hooting for unrested, birds lightly chirping for rested). In one or more embodiments, the user 101 may also be able set custom sounds that may be allocated to certain proportions of the sleep of the user 101. In one or more embodiments, the sufficiency of sleep may be communicated by verbalized words through the earphones 300, for example: “unrested,” “part rested,” or “full rested”; “uncharged” and “charged”; “still resting” and “ready”; and/or a verbalized percentage of the proportion. In one or more embodiments, the sleep sufficiency subroutine 242 may include computer readable instructions that when executed generate a sound representation of the proportion of the actual sleep duration value relative to the target sleep duration value including (i) a tone representing the proportion and/or (ii) a word describing the proportion. The sound representation may be played on the speaker 308 of the earphone 300.

In one or more embodiments, the sleep sufficiency, sleep proportion, and/or representations thereof may be hidden unless specifically requested by the user 101. The same may be true of a clock which can optionally be displayed on the display 204 of the coordination hub 200. For example, the user 101 may need to take an action to generate a query to receive the representation of a sleep proportion. In some cases, such an action might further wake the user 101 (e.g., fumbling for a cell phone, then touching a button such that the screen illuminates to display the time). However, in one or more embodiments, the earphones 300 may include a simple button and/or gestural interface that may include one or more touch input sensors on a portion of the earphones 300, such as an exterior facing portion (e.g., the touch interface 305). The user 101, when activating the gestural interface and/or button, may generate a query which resolves in generation of the graphical representation and/or the sound representation. One advantage of this approach may be that it may be easy for the user 101 to reach up to the earphones 300, rather than search for a specific device that could be in various locations. Another advantage may be that the light that might otherwise be emitted from a phone or bedside alarm clock is not emitted in favor of only temporary illumination when queried. In one or more embodiments, the coordination hub 200 includes a gesture agent 241 configured to receive a sleep level query from the earphones 300 of the user 101 and/or another source. In one or more embodiments, the gesture agent 241 may include computer readable instructions that when executed receive a sleep level query generated by a gesture input on a touch interface 305 of the earphone 300. Other queries are possible, for example a voice interface in which the user 101 may whisper “sleep check” or “how rested am I?”, and the coordination hub 200 may include an appropriate query receipt agent with voice recognition capabilities, as known in the art, to identify such query of the user 101 and generate a response.

In one or more embodiments, and as further shown and described in the embodiment of FIG. 6, a response to a query for a sleep level, sleep sufficiency, and/or sleep proportion may include other members within a sleep group 601. For example, for two sleeping spouses, the cognitive load reduction interface 205 may display a graphical representation of sleep proportion for both partners and/or a present cognitive state. An advantage may include that one member of the sleep group 601 may be able to determine if it would be detrimental to wake one or more of the other members of the sleep group 601. Certain queries may specifically only query the sleep level, sleep sufficiency, and/or sleep proportion of other members of the sleep group 601.

In one or more other embodiments, the sleep level, sleep sufficiency, and/or sleep proportion always may be displayed on a user interface for any user 101 actively sleeping, for example on the coordination hub 200. A form of display may also be integrated into the earphones 300, for example small lights on the exterior of the earphones 300 that represent the sleep level of the user 101 (e.g., green for rested, yellow for moderately rested, red for unrested), such lights being sufficiently dim as to not distract other sleeping members of the sleep group 601 and/or the user 101 wearing the earphones 300. In one or more other embodiments, sleep level, sleep sufficiency, and/or sleep proportion may be alternatively, or additionally, communicated and/or displayed on an app or other electronic display interface of one or more of the members of the sleep group 601. Similarly, one or more members of the sleep group 601 may receive notifications, alerts, and/or other communications related to the members of the sleep group 601 (e.g., a colleague is sufficiently rested and can now be awoken.

In one or more embodiments the coordination hub 200 may include and/or may have remote access to a database 250 (which may be stored on the memory 203 and/or a different computer readable memory). The database 250 may store the sleep session data 260 for one or more sleep sessions, the sleep map 500 for one or more sleep sessions, and/or one or more user profiles 252. It should be noted that, where the coordination hub 200 is designed to be utilized with a single user 101, the entire contents of the database 250 may represent the user profile 252. In contrast, where the coordination hub 200 may be utilized with two or more users 101, user profiles 252 may be designated for each (e.g., a user profile 252A, a user profile 252B, etc.).

The user profile 252 may include a user UID 253 that allows for the user profile 252 to be uniquely queried within all expected instances of the user profile 252 within a dataset. For example, where the user profile 252 may be also stored in a remote server (e.g., in a cloud server) alongside other user profiles 252, the user UID 253 may be a globally unique identifier (e.g., a GUID). The user profile 252 may include user data 254 (e.g., the name of the user 101, location information, etc.) and a set of associated devices 255 (e.g., that may be identified through a device UID 255A, a device UID 255B, etc.), for example earphones 300 and/or communication devices 400. The user profile 252 may include a set of event data 256 which may include one or more defined instances of disturbance events 102 which have designated priority levels and/or which are to be allocated to the user 101 associated with the user profile 252 during an active sleep group 601, as further shown and described in conjunction with the embodiments of FIG. 6.

The user profile 252 may include one or more sleep maps 500, as further shown and described in conjunction with the embodiments of FIG. 5A through FIG. 5E. The user profile 252 may also include one or more instances of sleep session data 260, for a present sleep session and/or historical sleep sessions. In one or more embodiments, the sleep session data 260 may include storage of data specifying the cognitive state of the user 101 over increments of time, for example every ten seconds, one minute, and/or ten minutes. The sleep session data 260 may also store data from which the cognitive state is derived and/or log disturbance events 102. The sleep session data 260 may be able to be later presented, visualized, and/or analyzed to assist in generating sleep statistics, historical sleep trends, assess recurring disturbances, and/or other useful information. Although two instances of the user profile 252 are illustrated in FIG. 2 (the user profile 252.1 and the user profile 252.2), it will be evident to one skilled in the art that an arbitrary number of user profiles 252 may be stored on the coordination hub 200.

In one or more embodiments, a temperature difference of the body temperature of the user 101 to the ambient temperature (e.g., the temperature the sleep environment 100, the temperature of the room in which the user 101 is sleeping, etc.) may assist in determining the cognitive state of the user 101. The coordination hub 200 may generate environment data 130 (not shown in FIG. 2, but illustrated in FIG. 3), for example an ambient temperature data 134. The coordination hub 200 may also generate environmental audio data 132 of the environmental sound 104, for example with the microphone 209.

In one or more embodiments, the network interface controller 202 may be configured to present the coordination hub 200 as a simple audio device that can be paired with through Bluetooth® or another local wired or wireless communication protocol such that almost any device that can pair to headphones or speakers can be interfaced with the coordination hub 200. For example, the coordination hub 200 may be pairable with a device generating a communication notification 108, where the coordination hub 200 is recognizable by the device as an earphone device through a pairing communication protocol (e.g., Bluetooth®). Sounds that would ordinarily be “passed” to the device can therefore be intercepted and evaluated before possible passthrough to the earphones 300. The disturbance classification engine 230, the sleep protection engine 220, and/or other components may therefore receive, evaluate, classify, and/or screen communications from devices that may not otherwise include such capabilities within its operating system (e.g., the operating system 406) and/or native applications.

FIG. 3 illustrates an earphone 300 and/or a set of earphones 300, according to one or more embodiments. It will be recognized by one skilled in the art that the earphones 300 may include an element in each earphone (e.g., a speaker 308A of an earphone 300A and a speaker 308B of an earphone 300B, for a right ear and left ear, respectively), or may include elements in only one of the earphones (e.g., a thermometer 318, the noise canceling system 307, etc.). The earphones 300 may take on any of a variety of form factors. For example, the earphones 300 may be earbuds, over-ear headphones, on-ear earphones, in-ear earphones, clip-on earphones, hearing aids, etc. In one or more embodiments, a pair of earphones 300, shown as the earphone 300A and the earphone 300B, may be wirelessly communicatively coupled through the network 140 to the coordination hub 200. In one or more preferred embodiments, the earphones 300 are wireless earbuds easily worn inside the ear such that a user 101 can sleep on their side without portions of the earphone 300 protruding from the ear of the user 101 or otherwise causing discomfort.

The earphones 300 may include a processor 301, a memory 303, a network interface controller 302, a power source 309 (e.g., a battery, a wired power supply), and/or a speaker 308. It will be recognized that miniature and/or combination processors 301 and/or memories 303 may be utilized, for example a QUALCOMM® QCC5171. The network interface controller 302 may include a wireless network interface controller that may communicate with one or more devices or systems over the network 140, for example the coordination hub 200, the other earphone 300 (e.g., the earphone 300A may be communicatively coupled to the earphone 300B) and/or the communication device 400.

The earphones 300 may include a low energy protocol 315 allowing for periodic, low-energy communication with the coordination hub 200 and/or other devices communicatively coupled to the network 140. The low energy protocol 315 may permit a smaller battery (e.g., an example of the power source 309) to be utilized for the earphone 300, which may further increase comfort of the user 101 earing the earphones, especially where the earphone 300 is an earbud intended to fit into and/or intended to be held in place in the ear canal of the user 101. The low energy protocol 315 may permit a small battery, and therefore earbud form factor, while still enabling enough battery life for a common full sleep period on a single battery charge (e.g., 8 hours, 10 hours, 12 hours). The low energy protocol 315 may transmit audio, or in one or more embodiments other data.

The noise canceling system 307 may include capabilities for masking noise, noise canceling, and/or white noise generation as may be known in the art, which may be collectively referred to as “external sound mitigation”. In one or more embodiments, the noise canceling system 307 may utilize feed forward active noise cancelation (e.g., feed-forward ANC), for example as provided through QUALCOMM® technologies.

The earphones 300 may include a touch interface 305, such as one or more buttons that can be depressed, switches that can be toggled, and/or a gestural interface. The gestural interface may sense a direction and/or speed of travel of a finger of the user 101, and/or a pressure of touch of the finger of the user 101. The direction, speed, and/or pressure may be associated with various controls such as volume, starting or ending noise canceling or masking, generating a sleep query (e.g., as shown and described in conjunction with the gesture agent 241 of FIG. 2), etc. In one or more embodiments, the touch interface 305 may be resistive detection points that may detect touch of human skin. In one or more embodiments, the touch interface 305 may be electrostatic sensors that may be an electrical potential sensing channel able to measure electrostatic potential change. Specifically, in one or more embodiments the electrostatic potential may be sensed utilizing an STMicroelectronics® ST Micro LSM6DSV16X.

The earphones 300 may include one or more sensors 310 for sensing physiological data 110 of the user 101. A microphone 312 embedded in an inner ear canal portion of the earphone 300 may receive audio from blood rushing through the ear of the user 101, audio of the user 101 breathing and/or audio of the user speaking, resulting in generation of the user audio data 112. An accelerometer 314 and/or a gyroscope 316 may result in accelerometer data 114 and/or gyroscope data 116. The accelerometer data 114 and/or the gyroscope data 116 may be analyzed to determine micro movements of the user 101 (e.g., respiration, heart beats) and/or macro movements of the user 101 (rolling over, changing position of the head, background movements associated with sleep on a public transportation vehicle, background movements associated with sleep in a hammock, etc.). The thermometer 318 may generate a user temperature data 118. Other sensors 310 may be integrated in addition to those specified. Optionally, certain sensors 310 may have two instances, one occurring in each of the earphone 300A and the earphone 300B, whereas other sensors 310 may occur in just one of the earphone 300A or the earphone 300B.

In one or more other embodiments, the microphone 312 may be exterior-facing, for example utilized to collect the environmental sound 104 and/or sounds of the user 101. Collection of the environmental sound 104 from a point of the earphones 300 may be advantageous in determining the environmental sound 104 as would be perceived by the ears of the user 101, rather than from the coordination hub 200. Such collection of audio at the earphones 300 may also assist in distinguishing sounds originating from one or more user 101 within a sleep group 601.

The earphones 300 may include an audio track 320 that may include masking audio, relaxing audio, and/or canceling waveforms. For example, the audio track 320 may include nature sounds, music, ambient sounds, and/or other sounds that may assist the user 101 in falling asleep. The audio track 320 may be streamed and/or downloaded through the network 140, and may be stored within the memory 303. The memory 303 may also receive and/or store a sound data 109 associated with a communication notification 108 and/or sound data 105 of an environmental sound 104, either of which may be played on the speaker 308. In one or more embodiments, the sound data 109 and/or the sound data 105 will have been transmitted to the earphones 300 following determination that an associated communication 106 and/or environmental sound 104, respectively, is of greater relative priority than the cognitive state and/or sleep period of the user 101. The earphones 300 may also generate environment data 130, for example environmental audio data 132 of the environmental sound 104, and/or ambient temperature data 134 (which may be generated by the thermometer 318 and/or an exterior facing thermometer 319, not shown).

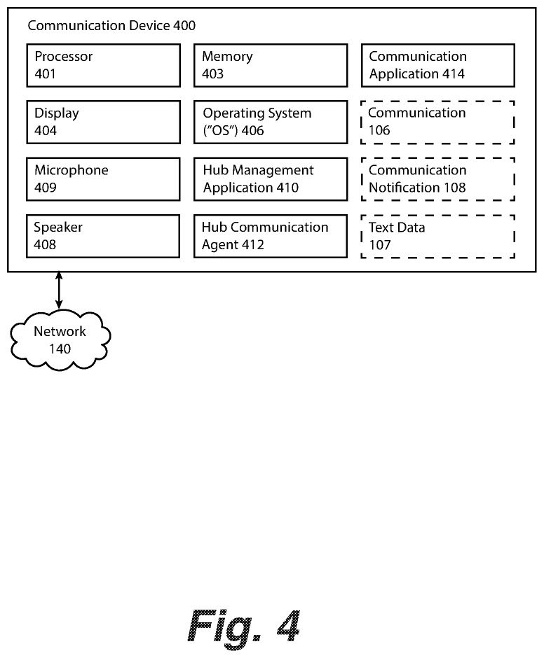

FIG. 4 illustrates a communication device 400, according to one or more embodiments. The communication device 400 may be any device which receives communications 106 and/or notifies the user 101 of communications 106, for example a smartphone, a mobile phone, a cellular phone, a landline phone, a pager, a tablet device, a laptop computer, a desktop computer, and/or a server computer. The communication device 400 may include a processor 401, a memory 403, a display 404 (e.g., indicator lights, a display screen, an LCD screen, an OLED screen, an LED screen), a speaker 408, and/or a microphone 409. The communication device 400 may include an operating system 406 (also referred to as the OS 406), for example Android®, iOS®, Microsoft Windows®, Linux®, Unix, MacOS®, ChromeOS®, etc.).

The communication device 400 may include a hub management application 410 that may allow the user 101 to configure the coordination hub 200, setup or edit data within the user profile 252, setup or edit sleep maps 500, view or analyze sleep session data 260, recording audio or managing profiles of the sound signature library 261, setting up sleep group profiles 272 and/or defining event data 256 (as further shown and described in conjunction with FIG. 6) and/or other functions. Depending on the communication device 400 and/or the operating system 406, the hub management application 410 may be a native application (e.g., an executable on a desktop computer, an app on a smartphone downloaded from an app store) and/or may be accessible as a plugin, API extension, or through a web browser. A hub communication agent 412 may be stored and executed on the communication device 400 to receive any communication back from the coordination hub 200, for example confirmation of transmission of a communication notification 108 and/or receiving a silencing command 266 that may be made as a result of a low-priority determination relative to the sleep of the user 101. The communication device 400 may further include the communication 106 (e.g., a text message, a call, an app notification, an email, a voicemail, a message generated by a server, etc.), the communication notification 108 (e.g., a push notification, a summary of the communication 106, a graphical badge indicating a communication 106 is pending, etc.). The communication 106 may include a text data 107, or the text data 107 may be generated from, extracted from, and/or otherwise derived from the communication 106. The communication 106 and/or the communication notification 108 may have originated from a communication application 414, for example a workplace chat app (e.g., Slack®), an encrypted messaging app (e.g., Telegram®), an email client (e.g., Outlook®), a video conferencing communication app (e.g., Zoom®), etc.

In one or more embodiments, there may be multiple instances of the communication device 400 communicatively coupled to the coordination hub 200. This may further assist in the classification and/or evaluation of communications 106 and/or communication notifications 108. As an example, where a user 101 has two smartphones, a call to the second smartphone may be determined to have an elevated priority level just after the same person called the first smartphone within a threshold time period (e.g., 1 minute).

FIG. 5A through FIG. 5E each illustrate a sleep map 500, according to one or more embodiments. Within the illustrated sleep maps 500 may be defined one or more time periods and/or cognitive states which may be placed in sequence, parallel with rules for which takes priority, in contingency, or overlaid. It will be recognized by one skilled in the art of computer programming and/or software development that the sleep maps 500 may be stored as data and need not be visualized as shown. It will be evident that visualizations have been provided for simplicity of explanation. In addition, graphical representation of the sleep map 500 as shown in FIG. 5A through FIG. 5E may be advantageous for the user 101, including during definition, assembly, and/or editing of a sleep map 500.

In general in the embodiment of FIG. 5A through FIG. 5E, a hexagon may indicate a period indicator 501 for a sleep period 502. “Hr” may designated a number of hours which may have elapsed from the beginning of the sleep map 500. “AM” or “PM” may designate a specific time, regardless of when the sleep map 500 begin. A diamond may indicate a state indicator 511 for a cognitive state 512. “PS” designates beginning of a pre-sleep state. “REM” designates the beginning of a random eye movement (REM) sleep state. “NREM” designates the beginning of a non-random eye movement (NREM) sleep state. Circled numbers may indicate priority values 520. A solid line around a rectangular section of time period 502 indicates that it may not be determined based on cognitive state, but rather time. In contrast, broken lines of various forms around the rectangle may designate a cognitive state period 512. Specifically. dotted lines may indicate the pre-sleep state 514, dot-dashed lines may indicate the REM state, and the dashed lines may indicate the NREM state. Where a rectangular portion of the sleep map 500 includes “+” infill, it may designate a high priority sleep period 504 and/or high priority sleep state.

FIG. 5A illustrates a sleep map 500A, according to one or more embodiments. The sleep map 500A may initiate when a first determination is made that the user 101 has achieved a REM sleep state, designated as the state indicator 511.1 to initiate the time period 502.1. (It should be noted that in the present example, the time period 502 need not include REM sleep once the REM state initiates the sleep map 504). The period from the state indicator 511.1 to the period indicator 501.2 may be a four-hour period which is designated as a high priority sleep period 504. Once the period indicator 501.2 is reached, the sleep period 502.2 may be initiated for a period of 3 hours until a period indicator 501.3 is reached, terminating the sleep session associated with the sleep map 500A. Upon termination, the user 101 may be allowed to wake naturally, may be woken by an alarm or other mechanism, and/or simply allowed to receive any disturbance events 102 without protection of the sleep protection network 150 and/or devices thereof. As a result, the user 101 may be able to (i) ensure they fall into deep, high value sleep prior to initiation of the timeline associated with the sleep map 500, (ii) ensure at least four hours of high priority sleep is achieved, and (iii) receive up to seven hours of sleep provided no high priority disturbance events 102 need to interrupt the sleep of the user 101.

FIG. 5B illustrates another example of a sleep map 500B, according to one or more embodiments. In contrast to the sleep map 500A, the sleep map 500B may instead track consecutive sleep states to determine sufficiency of the sleep of the user 101, according to one or more embodiments. The state indicator 511.1 may initiate in pre-sleep state, resulting in the cognitive state period 512.1. The second cognitive state period 512.2 (not labeled) may be initiated upon the second state indicator 511.2 (not labeled), shown as the NREM indicator and initiating the REM state 518A. The third cognitive state period 512.3 (not labeled) may be initiated upon the third state indicator 511.3 (not labeled), shown as the REM indicator and initiating the REM state 518A. The fourth cognitive state period 512.4 (not labeled) may be initiated upon the second state indicator 511.4 (not labeled), shown as the NREM indicator and initiating the NREM state 516B. Finally, the fifth cognitive state period 512.5 (not labeled) may be initiated upon the fifth state indicator 511.5 (not labeled), shown as the REM indicator and initiating the REM state 518B.

FIG. 5C illustrates a hybrid between mapping time periods 502 and cognitive state periods 512. Specifically, the sleep map of FIG. 5C may begin at upon the period indicator 501.1 (e.g., at 11 PM, regardless of the cognitive state of the user 101). By default, the time period 502.1 (not labeled in FIG. 5C) between 11 PM and 3 AM may be a high priority sleep period 504 (not labeled). Sometime between 11 PM and 3 AM, if the user 101 starts a REM sleep state (e.g., to result in REM cycle completion 515), then the high priority sleep period 504 may extend past 3 AM and into the second sleep period 502.2 (not labeled), taking priority over the low priority sleep period 506 (not labeled in FIG. 5C) until the REM cycle completion 515. FIG. 5C illustrates that a communication 106 and/or a communication notification 108 may have been received during the high value sleep period 504, but delivered following termination of the REM state and/or the high value sleep period 504.

FIG. 5D illustrates another hybrid between mapping the time periods 502 and the cognitive state periods 512. Similar to the previous example, the sleep map 500D of FIG. 5D may begin at the period indicator 501.1 (e.g., at 11PM, regardless of the cognitive state of the user 101). By default, the time period 502.1 (not labeled in FIG. 5C) between 11 PM and 3 AM may be a high priority sleep period 504 (not labeled). Sometime between 11 PM and 3 AM, if the user 101 starts and finishes a REM sleep state (e.g., to result in REM cycle completion 515), then the high priority sleep period 504 may be converted to and/or downgraded to a low priority sleep period 506 (not labeled in FIG. 5C).

FIG. 5E illustrates a sleep map 500E that includes numerical priority values 520 and/or overlay periods 522. The sleep map 500E of FIG. 5E may begin with the state initiator 511.1 (not labeled) by a sleep state of the user 101 and proceed to the period initiator 501.2 (not labeled) at a four-hour elapse time from the state initiator 511.1 to form a four-hour sleep period. During the period from the state initiator 511.1 to the period initiator 501.2, a base priority value 520.1 of ‘3’ may be associated. The period initiator 501.3 may occur at five hours from onset of the state initiator 511.1. From the period initiator 501.2 to the period initiator 501.3 may be a period of one hour with an assigned priority value 520.3 of ‘2’. Finally, the period initiator 501.4 may occur at seven hours elapse time from the period initiator 501.1, forming a two hour period between the period initiator 501.3 and the period initiator 501.4, to which a priority value 520.5 of ‘1’ may be associated.

The sleep map 500E may then include numerous overlays to increase (and/or decrease) the priority level. As illustrated, the state initiator 511.1 may initiate a REM state which terminates at the state initiator 511.2 designating an NREM state, where the period of the REM state results in an increase in the priority value 520.2 of ‘+1’. The REM state may form an overlay period 522. For example, where the user 101 is both within the initial four hours from the initiator 501.1, and within the overlay period 522, the sleep priority value would be three, plus two, for a total of five. Similarly, a second REM period, the overlay period 522B, will result in ‘+2’ within any overlayed time period. The sleep map 500E therefore illustrates a variation in sleep priority levels from one to five that may be utilized to determine whether disturbance events 102, ranked according to the same scale, should be permitted to disturb the user 101. A high priority sleep period 504 can be optionally and arbitrarily designated at a given value on the scale, for example at a ‘3’. In one or more other embodiments, it will be evident to one skilled in the art that non-integers, larger scales, and/or weighted multidimensional sleep factors may be utilized (e.g., quality of sleep appears high, and the user 101 has not experienced quality sleep in several days, thereby further increasing the priority value 520 of an overlay period 522 of REM sleep).

FIG. 6 illustrates a group sleep network 650, according to one or more embodiments. In one or more embodiments, and the embodiment of FIG. 6, the coordination hub 200 may be utilized to improve the sleep of a group of users 101, either individually or collectively. The group may be referred to as the sleep group 601 or sleep group 601. The sleep group 601 may be small, for example two instances of the user 101 (e.g., a user 101.1, a user 101.2), and the coordination hub 200 may be therefore customized for use by such two users 101 (e.g., a set design and marketing for spouses). In one or more other embodiments, the sleep group 601 may encompass three or more family members. For example, two spouses and an infant child, where sensors (e.g., similar to the sensors 310 of FIG. 3, but which may not be incorporated into earphones 300) may provide additional data and information to the coordination hub 200. In one or more other embodiments, the sleep group 601 may be larger and occur in a commercial, industrial, and/or military environment. For example, the sleep group 601 might be two or more members of a merchant marine crew piloting a container ship, firefighters operating in shifts on a forest wildfire, operators of an off-shore oil rig, operators of a spacecraft communicating at various ground stations located around earth, and/or military personnel. Although shown within the same sleep environment 100, the users 101 may be located in different sleep environments 100 (e.g., a sleep environment 100.1 of the user 101.1, etc.).

In one or more embodiments, the coordination hub 200 may include a sleep grouping subroutine 236 configured to setup and configure a sleep group 601 and an associated sleep group profile 272. In one or more embodiments, the sleep grouping subroutine 236 may include computer readable instructions that when executed associate a device ID (e.g., an instance of the device UID 255) of the first earphone 300.1 (e.g., comprised of the earphone 300.1A and the earphone 300.1B) with a user profile 252.1 of the first user 101.1 and associate a device ID (e.g., an instance of the device UID 255) of a second earphone 300.2 (e.g., comprised of the earphone 300.2A and the earphone 300.2B) with a user profile 252B of a second user 101.2. The sleep grouping subroutine 236 may also include computer readable instructions that when executed designates a sleep group profile 272 that includes the user profile 252.1 of the first user 101.1 and the user profile 252.2 of the second user 101.2. The user profile 252.1 and/or the user profile 252.2 may include any of the data shown in FIG. 2, and/or additional data. As illustrated in the present embodiment, the sleep group profile 272 includes at least each include an instance of a unique identifier 253 and a sleep session data 260. The sleep grouping subroutine 236 may store the sleep group profile 272 as a collection of user profiles 252 and/or references thereto through unique identifiers, for example data attributes storing the user UID 253.1 and the user UID 253.2 in the present example.

In one or more embodiments, an event designation subroutine 338 may be configured to designate disturbance events 102 or other events that should be allocated to a given user profile 252, an order for priority of designation (e.g., first the user 101.2, and if no response, then to the user 101.1), and/or circumstances of allocation. The disturbance event 102 or other event to be allocated to a user profile 252 may be stored in the data structure of the sleep group profile 272 as the event 274, including optional association with the user UID 253 and/or the user profile 252 grouped within the sleep group profile 272.

In one or more embodiments, the event designation subroutine 338 includes computer readable instructions that when executed define event data, and assign the event data to the user profile 252 of one of the users 101 in the sleep group 601, for example the user profile 253.2 of the second user 101.2.

In one or more embodiments, the disturbance classification engine 230 may be configured to match a disturbance event 102 (e.g., an environmental sound 104, a communication 106), for example following classification, to an event data 274. The disturbance classification engine 230 may include computer readable instructions that when executed receive a disturbance event 102, and determine the disturbance event 102 is defined in the event data 274. In the present example, it may be determined that the disturbance event 102 matches the event data 274.2B. The coordination hub 200 may include a group allocation subroutine 226 that is configured to determine the user profile 252 associated with the event data 274 that may have been determined to be a match by the disturbance classification engine 230. In one or more embodiments, a group allocation subroutine 226 includes computer readable instructions that when executed query the sleep group profile 272, the user profile 252A of the first user 101.1, and the user profile 252B of the second user 101.2 to determine which instance of the user 101 is associated with the event data 274. In the present example, it may be determined that the user profile 252.B is associated with the event 274.2B.

In one or more embodiments, the sleep protection engine 220 may be configured to protect the sleep of one or more of the users 101 within the sleep group 601 by allocating the disturbance event 102 to one or more other users 101 within the sleep group 601. In the present example, the disturbance event 102 matching the event data 274.2B may be allocated to the user 101.2. The allocation may occur, for instance, by causing a sound such as a chime or alarm in the earphones 300.2 of the user 101.2. In one or more embodiments, the sleep protection engine 220 may include computer readable instructions that when executed generate an audio indicator of the event data 274 on the earphone 300 of the associated user 101 (e.g., the audio indicator may include, for example, the sound data 109 associated with the communication notification 108, the sound data 105 associated with the environmental sound 104, and/or other sounds). Other indicators (e.g., vibration, lights) may also be utilized to allocate the disturbance event 102.

As a result, disturbance events 102 may be matched and allocated to individual users 101 and/or subsets of users 101 within the sleep group 601. Priority for allocation may also be specified within the event data 274 and/or the sleep group profile 272. As just one example, a crying infant may first be allocated a user 101.1 who is a mother, second to a user 101.2 who is a father, and third to a user 101.3 who is a sibling. As another example, certain notifications from a server computer may be routed to certain individuals within the sleep group 601, for example a connectivity issue to a first user 101.1 and a hard disk failure error to a second user 101.2. Certain event data 274 may also be defined such that they are directed to all users 101 within the sleep group 601 to ensure high priority emergencies are responded to, even at the expense of the sleep of the entire sleep group 601. This may also help to entire group to sleep knowing emergencies will be responded to even if some of the group were to sleep through an alarm or other intentional wake-up procedure.

In one or more embodiments, the group sleep network 650 may also maximize sleep of the group by allocating according to how well rested a the users 101 of the sleep group 601 are, according to group or individualized sleep maps 500, and/or according to cognitive states of the users 101 of the sleep group 601.

In any of the embodiments, sleep priority level may also be based on an aggregate sleep and/or restfulness score, in which total restfulness may be valued by adding, multiplying, and/or applying weighted factors to various sleep periods (e.g., 1 point per hour of NREM, 2 points per hour of REM, etc.).

In one or more embodiments, the event designation subroutine 338 may be configured to receive a disturbance event 102, query the sleep session data 260 of each user 101 within the group, and allocate the disturbance event 102 to a user 101 that is the most rested. In one or more embodiments, the event designation subroutine 338 may be configured to receive a disturbance event 102, query the sleep session data 260 of each user 101 within the group, and allocate the disturbance event 102 to a user 101 that is in a lowest priority sleep level (e.g., NREM sleep).

Allocation may also be based on a combination of approaches. For example, an event data 274 may be defined and associated with two out of five users 101 within a sleep group 601. When the event data 274 is matched to a disturbance event 102, and both the user 101.1 and the user 101.2 are determined to be associated with the event data 274, then allocation can be based on the cognitive state of each of the users 101.1 and 101.2 (e.g., allocation to 101.1 where the user 101.1 is more rested than the user 101.2).

In one or more embodiments, the cognitive load reduction interface 205 may support presentation of the sleep levels and/or proportions of one or more members of the sleep group 601. For example, when a sleep sufficiency query is generated by the user 101.1 (e.g., as may be detected by the gesture agent 241), a graphical representation and/or sound representation of the proportion of the sleep of both the user 101.1 and the user 101.2 may be provided to the user 101.1. This may assist in helping the user 101.1 determine whether it is appropriate to wake the user 101.2 and/or how careful the user 101.1 should be to protect the sleep of the user 101.2. In one or more embodiments, sleep levels, cognitive states, and/or sleep proportions may be able to be viewed by administrative, managerial, and/or other personnel or members of the sleep group 252 to assist in monitoring the sleep health, restfulness, and/or readiness of the sleep group 601.

In one or more embodiments, sound and/or movement (such as macro movement) detected as originating from one user 101 of a sleep group 601 may generate a response in one or more of the other earphones 300 of one or more other users 101 of the sleep group 601. As an example, where the microphone 312 of a earphones 300A detects sound emanating from a user 101A (e.g., snoring, rustling sheets, a creaking door) and/or accelerometer or IMU gathers data indicating macro movement (e.g., getting out of bed, walking), the sound masking volume of the earphones 300B for a user 101B may be increased in response. This may allow for limited use of increased masking volume in response to known events of other users 101 within the sleep group 601, where indiscriminate and/or persistent use of masking volume may otherwise bother or cause the user 101 to awaken.

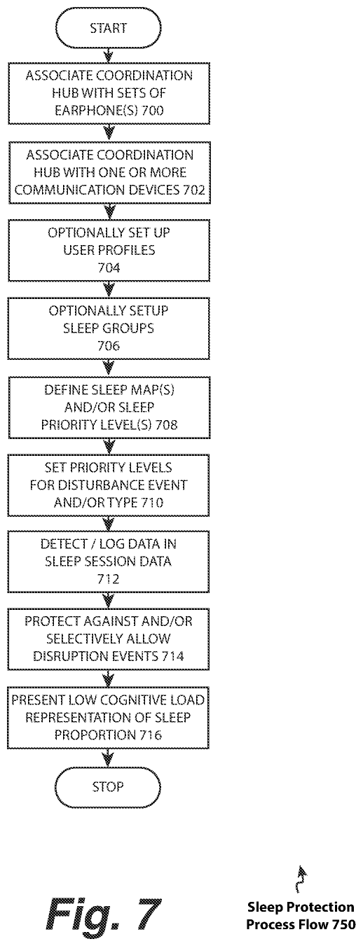

FIG. 7 illustrates a sleep protection process flow 750, according to one or more embodiments. Operation 700 associates a coordination hub 200 with a set of earphones 300. The association may occur in a computer readable memory, for example a database. The association may occur through a communication pairing (e.g., over the network 140) through network address storage and/or through storage of a device UID (e.g., the device UID 255), such that the set of earphones 300 may be communicatively coupled and/or ‘recognized’ by one or more systems, routines, and/or modules of the coordination hub 200.

Operation 702 may associate the coordination hub 200 with one or more communication devices 400. Similarly to operation 700, the association may occur through a communication pairing (e.g., over the network 140) and/or through storage of a device UID (e.g., the device UID 255), such that each of the communication devices 400 may be communicatively coupled and/or ‘recognized’ by one or more systems, routines, and/or modules of the coordination hub 200.

Operation 704 may optionally set up one or more user profiles 252, including assigning unique identifiers to each (e.g., the user UID 253) such that each can be individually addressed within a database (e.g., the database 250, the database 270, a different database). The setup may include receiving data from one or more users 101, assigning device UIDs 255 associated with the user profile 252, inferring data (e.g., sleep patterns, noise common to the sleep environment 100 of the user 101, etc.), and manual and/or custom configuration. Operation 706 may optionally define sleep groups 601 of one or more users 101 that may have related interests in sleep, sleep sufficiency, and/or coordinated sleep patterns. For example, a sleep group 601 may be designated with a sleep group profile 272 that may be a list of user UIDs 253 of user profiles 252 to be included within the sleep group 601.

Operation 708 may define sleep maps 500 and/or set sleep priority levels. For example, a sleep map 500 may be initiated in memory as a set of data attributes that may include: a starting condition for the sleep map 500 that may initiate a first period, a starting condition for a second period of the sleep map 500, and a termination condition to end the sleep map 500. The sleep map 500 may also include data attributes and values specifying a priority level for each period, whether numerical or otherwise. Operation 708 may define Boolean logic related to the sleep map 500 (e.g., IF the user 101 is determined to be in a REM state, THEN maintain a high priority sleep value, ELSE drop to a low priority sleep value). Alternatively, or in addition, operation 708 may set a sleep priority level for the user 101, for example during a sleep state, during a REM state, during a time period, etc.

Operation 710 may set a priority level for one or more disturbance events 102 and/or types of disturbance events 102. For example, a disturbance event 102 may be defined as a phone call (e.g., a communication 106), a phone call from a specific type of person (e.g., a person associated with work, a family member), a phone call with seeming impatience and/or urgency (e.g., a phone call that calls more than three times, or calls multiple instances of the communication device 400), a phone call within a specific time period from a specific person (e.g., a coworker on Sunday), etc. Many other disturbance events may be defined, even those generated and communicated to the coordination hub 200 from an API. For example, and to demonstrate flexibility of the one or more of the present embodiments, a first disturbance event 102 that may be a low priority is a stock falling 3% during trading hours, while a second disturbance event 102 that may be a high priority may be a stock falling 10% during trading hours or 5% within one hour. Other examples of disturbance events 102 may include data generated by a smart home, for example a door bell ring, a home alarm or motion sensor activation, etc.

Operation 712 may detect and/or log data in a sleep session data 260. The data in the sleep session data 260 may include a cognitive state of the user 101 throughout a sleep period in periodic increments. The cognitive state may be determined through a variety of devices and methods, including as shown and described herein. In one or more embodiments, cognitive state of the user 101 and/or sleep state of the user 101 may be determined through one or more of respiration rate, heart rate, heart rate variability, temperature of the user 101, temperature change of the user 101 over time, temperature of the user 101 relative to an ambient temperature of the sleep environment 100, and/or other factors. The cognitive state may also be determined based on combing factors. For example, macro movement of the user 101 may override any other indication that the user 101 may be asleep, and/or certain pairing of respiration and heart rate variability may indicate REM sleep.

Operation 714 protects against and/or selectively allows disturbance events 102. Operation 714 may evaluate the importance, urgency, and/or priority of a disturbance, such as a communication 106 and/or environmental sound 104, and then determine whether the disturbance event 102 should “get through” to the user 101, should be stored for later delivery at a more advantageous time period (to the sleep of the user 101), and/or should be stored without further action. Operation 712 may also protect against a disturbance event 102 from one user 101.1 within a sleep group 601 by allocating the disturbance event 102 or notification thereof to a different user 101.2 within the sleep group 601.

Operation 716 presents a low cognitive load representation of sleep sufficiency, for example a proportion of sleep that is the actual sleep of the user 101 (and/or actual amount of a certain cognitive state, such as REM sleep) relative to a target amount of sleep of the user 101. The low cognitive load representation may include a graphical representation that quickly permits the user 101, without resorting to reading or character analysis, to determine the sufficiency of sleep. For example a set of emojis (e.g., gradations of sad and happy, gradations of sleepy and awake faces, a “sleep battery” graphic, a sufficiency color, etc.). The representation may also include a sound representation, for example spoken words, tones, songs, soundbites, and/or other audible indicators.

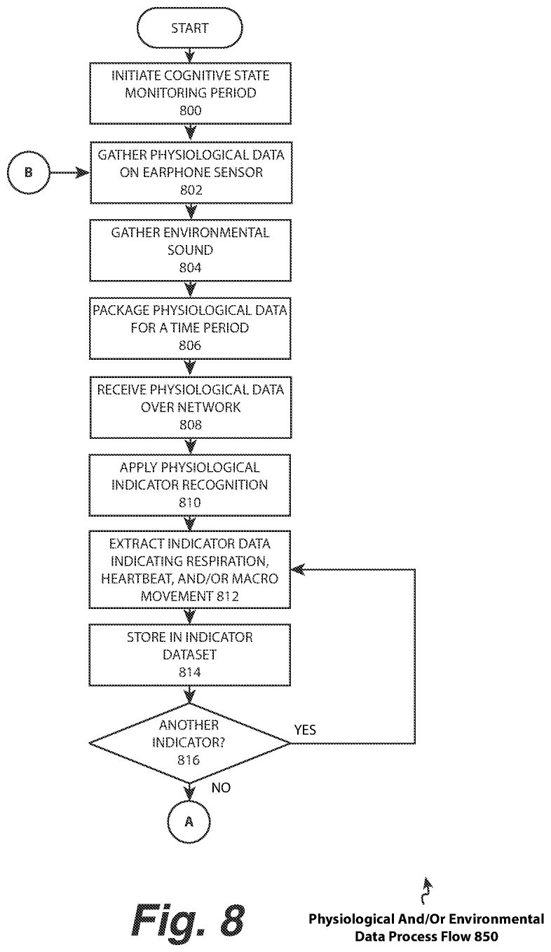

FIG. 8 illustrates a physiological and/or environmental data process flow 850, according to one or more embodiments. Operation 800 may initiate a cognitive state monitoring period of a user 101. The cognitive state monitoring period may be a period of time for collection of physiological data 110 of a user 101 to determine cognitive state. In one or more embodiments, initiation of the cognitive state monitoring period may occur automatically, for example when the user 101 removes earphones 300 from a changing case (e.g., which may be integrated with the coordination hub 200), when the user 101 fits the earphones 300 to the ear of the user 101 as may be determined from one or more sensors 310 of the earphones 300, etc.

Operation 802 gathers a physiological data 110 from a sensor 310 of the earphone 300. For example, the sensors 310 may include one or more microphones 312 acoustically coupled with the inner ear of the user 101, an accelerometer 314, a gyroscope 316, a thermometer, 318, a different type of motion sensor (e.g., inertial measurement unit, or IMU), a vibration sensor, etc. The physiological data 110 may include, for example, a user audio data 112, an accelerometer data 114, a gyroscope data 116, a user temperature data 118, a motion data, a vibration data, and/or other data. Operation 804 may gather an environmental temperature, for example from a sleep environment 100 of the user 101. The environmental temperature may be gathered from the thermometer 318 (provided it is configured such that the user 101 body temperature does not result in a inaccurate reading), a thermometer of the coordination hub 200, a thermometer of a communication device 400, and/or another thermometer. Operation 806 packages physiological data 110 for a time period, for example 50 milliseconds, 500 milliseconds, 1 second, and/or 10 seconds. Operation 808 receives the physiological data 110 (e.g., each package thereof) over a network 140, for example a local area network, a Bluetooth® network, etc.

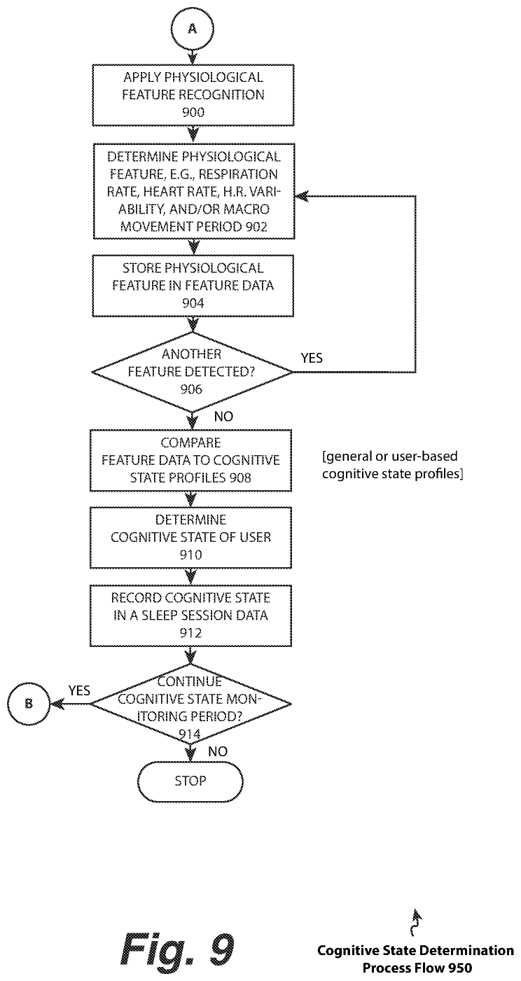

Operation 810 applies a physiological indicator recognition process to determine a physiological indicator within the physiological data 110. For example, certain small and recurring movements of the user 101, as may be determined through the accelerometer data 114, may indicate respiration events of the user 101. Operation 812 may then extract indicator data indicating respiration, heartbeat, and/or macro movement of the user 101. The extracted data may be stored as the indicator data 120. The indicator data 120 may be stored in an indicator dataset for further analysis, for example feature extraction as shown and described in the process flow of FIG. 9.

Operation 816 determines whether there is another indicator for extraction. If there is another indicator for extraction, operation 816 returns to operation 812 which may extract the indicator data, and subsequently add the indicator data 120 to the indicator dataset in operation 814. If no additional indicators are to be extracted, operation 816 may proceed along path ‘circle A’ to operation 900 of FIG. 9.

FIG. 9 is a cognitive state determination process flow 950, according to one or more embodiments. The cognitive state determination process flow 950 may either initiate at operation 900, or may be a continuation of FIG. 8 along path ‘circle A’. Operation 900 may apply a physiological feature recognition process, for example to the indicator data 120. As an example of physiological feature recognition includes evaluating one or more instances of the indicator data 120 to aggregate individual physiological indicators into a feature, for example heart beats into a heart rate, heart beats and/or heart rate into a heart rate variability, and/or breaths or respiration events into a respiration rate.

Operation 902 determines a physiological feature 122 is present within the indicator data 120, for example a respiration rate, a heart rate, a heart rate variability (abbreviated “H.R. variability” in FIG. 9), and/or a macro movement periods of the user 101. Operation 904 stores the physiological feature 122 in a feature data 124. As an example, the feature data 124 may include data attributes and values that store data representing the physiological feature 122 over a period of time. The period of time may be arbitrarily set depending on a desired resolution of cognitive state determination. For example, where the period is one minute, subsequent cognitive state determination may occur at a resolution of one minute or more. In one preferred embodiment, the resolution is thirty (30) seconds. Operation 904 stores the physiological feature 122 in the physiological data 110. The physiological data 110 may be stored in a computer readable memory (e.g., the memory 203, the memory 403), including optionally in association with the user profile 252 of the user 101 from which the physiological data derives. Operation 906 determines whether another physiological feature is present, in which case operation 906 returns to operation 902, and if not, proceeds to operation 908.

Next, a cognitive state may be determined from the feature data 124. In one or more embodiments, cognitive state may be determined by operation 908 comparing the feature data 124 and/or physiological features 122 of the feature data 124 to a cognitive state profile 264. The cognitive state profile 264 may include rules relating physiological features to cognitive states, signatures of states to be matched against, and/or indicators of various cognitive states. In a straightforward example, the cognitive state profile 264 may include a ruleset, such that the user 101 may be experiencing a sleep state where the respiration rate is a distance below a baseline respiration rate of the user 101, and the heart rate is distance below a baseline heart rate. In another example, heart rate trends and/or respiration rate trends may be assessed to determine the cognitive state. For example, respiration rate may decrease as a user begins to fall asleep (e.g., a pre-sleep state) followed by a rapid increase in respiration rate following sleep onset (e.g., the sleep state). In such example, an increase in rate according to and/or fitting a predetermined profile may result in determination of the cognitive state and/or transition between cogitative states. In one or more embodiments, the cognitive state profile 264 may be user-specific. In one or more other embodiments, the cognitive state profile 264 may include training data and/or model modification data that may modify, finetune, and/or enhance a machine learning algorithm and/or artificial neural network applied by the cognitive state determination routine 216.

Operation 910 determines the cognitive state of the user 101, for example by utilizing the cognitive state profiles 264. Where two or more physiological features in the feature data 124 may result in contradictory cognitive state determinations, priorities and/or additional rules may be utilized to reconcile and/or finalized cognitive state determination. Where a probabilistic model of state determination is used, probabilities of a given state above a certainty threshold may be used. Operation 912 records a cognitive state in a sleep session data 260. The sleep session data 260 may be stored as a data structure with sequential time entries each representing a time period, each entry including the cognitive state (and/or cognitive states probabilities), and/or backup data (e.g., the feature data 124 or portion thereof giving rise to the cognitive state determination). Other data that may be useful for later analysis may also be stored, for example occurrence and/or data logs of disturbance events 102 such as communications 106 and/or environmental sounds 104.

Operation 914 determines whether to continue the cognitive state monitoring period. If monitoring is to continue, operation 914 may return to operation 802 of FIG. 8 along path ‘circle B’. If the cognitive state monitoring period is to end, operation 914 may proceed to terminate. Operation 914 may make the determination based on one or more factors, including a length of time of an awake state of the user 101, a time of day (e.g., past 8 AM), whether the user 101 has completed all portions of a sleep map 500, and/or a combination of any of these factors (e.g., an awake state for more than 2 minutes anytime after 6 AM).

It will be recognized to one skilled in the art of computer programming and/or software development that the environmental data process flow 850 and the process flow 950 may run concurrently, for example physiological data 110 being generated and transmitted by one device or process, while the feature data 124 may be generated and/or cognitive state determination made by another device or process. In such case, physiological data may be buffered in computer memory pending evaluation and/or feature extraction. For example, the earphone 300 may generate and transmit physiological data 110 and the feature data 124 may be generated and/or cognitive state determination made by the coordination hub 200, according to one or more embodiments.

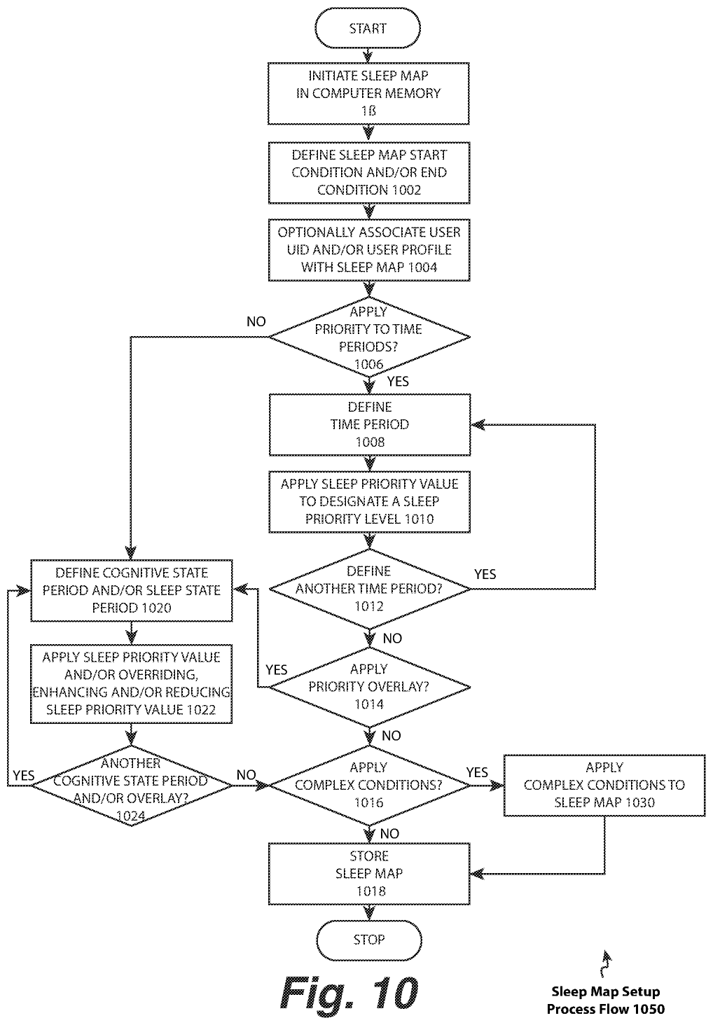

FIG. 10 illustrates a sleep map assembly process flow 1050, according to one or more embodiments. Operation 1000 initiates a sleep map in computer memory. The sleep map 500 may include data attributes that are usable, when paired with data values, to specify an intended sleep routine and/or sleep program for a user 101, for example as shown and described in conjunction with the embodiments of FIG. 5A through FIG. 5E. The sleep map 500 may be provided with a unique identifier, and/or a human readable name (e.g., “standard workday nigh”, “quick nap”, “long nap”, “migraine recovery sleep”, etc.).

Operation 1002 may define a sleep start condition and/or end condition for the sleep map 500. The start condition may be manual (e.g., the user 101 provides a start gesture to a touch interface 305), automatic based on interaction with a device (e.g., placement of the earphones 300 in the ear of the user 101), based on time (e.g., 9 PM occurs), based on detection of cognitive state either instantaneously or for a prolonged period (e.g., the user 101 enters a pre-sleep state, sleep state, or REM state), and/or physiological features (lack of macro movement for more than 5 minutes). Similarly, the end condition may be manual, automatic based on interaction with a device (e.g., removal of the earphones 300, or removal for more than 10 minutes), based on time (e.g., past 11 AM), and/or detection of a cognitive state instantaneously or for a prolonged period, and/or physiological features (e.g., macro movement or walking movement for more than 2 minutes).

Operation 1004 optionally associates a user UID 253 and/or user profile 252 with the sleep map 500. For example, a default instance of the sleep map 500 may be selected by the user 101, or the sleep map 500 being defined may be a custom sleep map 500 being set up by the user 101. Operation 1006 determines whether to apply a priority to a time period of the sleep map 500. If time periods are to be defined, operation 1006 may proceed to operation 1008. Otherwise, if a time period is not to be utilized, operation 1006 may proceed to operation 1020.

Operation 1008 defines a time period. For example, a 4 hour time period may be selected (e.g., the time period 502.1). Operation 1010 may then apply a sleep priority value to designate a sleep priority level. For example, the sleep priority value may be a numerical value, a binary (e.g., ‘0’ for low priority, ‘1’ for high priority), text data indicating priority level, etc. Operation 1012 determines whether another time period should be defined (e.g., a sleep time period 502.2), in which case operation 1012 returns to operation 1008. If no additional sleep period is to be defined, operation 1010 may proceed to operation 1014.

Operation 1014 determines whether a priority overlay that may be based on cognitive state should be applied to one or more of the time periods, in which case operation 1014 may proceed to operation 1020, and otherwise proceed to operation 1016. Returning the present explanation to operation 1006, where a time period is not to be selected, operation 1006 may also proceed to operation 1020 for definition of one or more cognitive state periods.

Operation 1020 may define a cognitive state period (such as a sleep state period), for example to define a cognitive state period 512.1. Operation 1022 may apply a sleep priority value to the cognitive state period. Where operation 1022 as been arrived at through operation 1014, as described below, operation 1022 may instead apply an overriding value (changing the priority value from a ‘2’ to a ‘4’), a multiplying value (e.g., 80%, 110%, a 2× multiplier), an enhancing value (e.g., adding ‘+2’ to a priority value), and/or a reducing value (e.g., subtracting ‘−1’ from a priority value). Operation 2024 determines whether an additional cognitive state period should be defined (or in the case of arrival from operation 1014, an overlay), in which case operation 1024 returns to operation 1020. If not, operation 1024 may proceed to operation 1016.

Operation 1016 determines whether to apply one or more complex conditions to the sleep map 500, in which case operation 1016 may proceed to operation 1030 which may apply the one or more complex conditions to the sleep map 500. For example, a condition may be defined whereby if a cognitive state period (e.g., a REM state) lasts more than 4 hours, a priority value may be decreased for the cognitive state period. In another example, a complex condition may be to determine a sleep priority value based on a calculated restfulness of a previous sleep period and/or cognitive state period. For instance, if a user 101 achieved a four hour REM cycle, a second REM cycle has a reduced enhancing value (e.g., +1 instead of +2). In yet another example, a complex condition may include that a sleep priority value diminishes as a function of time (e.g., a variable t) and/or as a function of sleep quality (e.g., a variable q). Operation 1030 may then proceed to operation 1018, which may also be arrived at from operation 1016 should no complex conditions need to be defined. Operation 1018 may then store the sleep map 500, for example in association with the user profile 252.

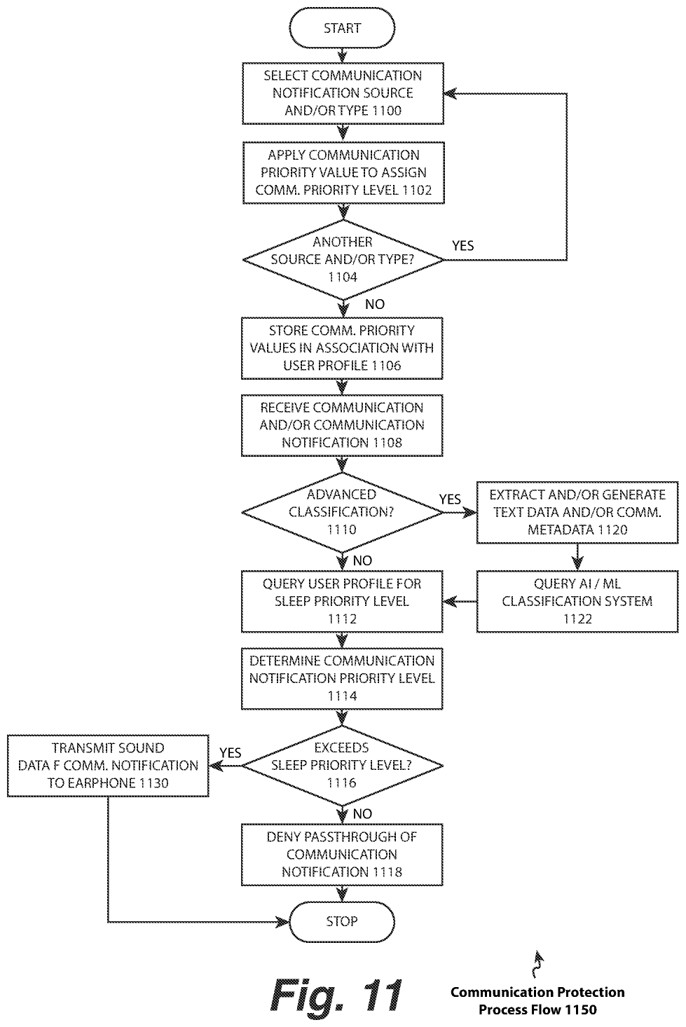

FIG. 11 illustrates a communications protection process flow 1150, according to one or more embodiments. Operation 1150 selects a communication notification 108 source and/or type. For example, the communication notification 108 source may be a communication device 400, individual communications 106 or senders thereof, categories of communication (e.g., notices from messaging apps, notices from productivity apps), etc. Operation 1102 applies a communication priority value to assign a communication priority level. For example, the communication priority value may be a numerical value, a binary (e.g., ‘0’ for low priority, ‘1’ for high priority), text data indicating priority level, etc. Designations may also be conditional, such as a ‘low’ priority unless the communication 106 is repeated on a different channel (e.g., a phone call, then a text message), or ‘high’ unless a text data 107 includes words indicating the communication 106 is ‘not urgent’ or references a timeframe more than one day away (e.g., ‘next Tuesday’). Operation 1104 determines whether another source and/or type is to be defined, in which case operation 1104 may return to operation 1100. Operation 1104 otherwise proceeds to operation 1106. Operation 1106 may store one or more communication priority values in association with a user profile 252. Alternatively or in addition, the communication priority values defined in the process flow of FIG. 11 may be default values applicable to more than one user 101.

Operation 1108 receives a communication 106 and/or a communication notification 108, which may be a disturbance event 102 that may disturb the sleep of the user 101. Operation 1110 determines whether an advanced classification is to be applied. Where an advanced classification is to be utilized, operation 1110 ma proceed to operation 1120 which may extract and/or generate a text data (e.g., the text data 107) and/or extract metadata related to the communication 106. Operation 1122 may then query an artificial intelligence and/or machine learning classification system, for example a large language model trained to determine an urgency and/or importance of messages based on training data. Feedback during a supervised learning process may, for example, receive feedback from one or more human subjects that classify whether various messages, from various contacts and with various message contents, would result in the user 101 wishing to receive the message even if it would disturb the user 101 from sleep. The training process of the AI/ML system may also include feedback from actual users 101, for example providing a list of communications 106 and/or communication notifications 108 received and withheld from the user 101 while sleeping, and allowing the user to review and provide feedback as to whether the user 101 actually wished to be disturbed. Conversely, the user 101 may provide feedback on communications 106 and/or communication notifications 108 that were allowed to disturb the user 101. Although text data 107 is specified for analysis, in one or more embodiments tone of voice, or other sound qualities may be assessed for classification.

Where advanced classification is not utilized, operation 1110 may proceed to operation 1112 which may query the user profile 252 (or other storage location) to determine the sleep priority value. Operation 1114 may then determine a communication priority level based on the communication priority value stored in operation 1106 and/or as a result of the advanced classification as the output of the AI/ML system queried in operation 1122.

Operation 1116 determines whether the communication priority level exceeds the sleep propriety level. If the communication priority level exceeds the sleep priority level, operation 1116 proceeds to operation 1130 which may transmit a sound data of the communication notification 108 associated with the communication 106 (e.g., a text arrival chime, a phone ring) to the earphone 300 of the user 101. If the communication priority level does not exceed the sleep priority level, operation 1116 may proceed to operation 1118 which may deny passthrough of the communication notification 108 associated with the communication 106. The communication 106 and/or the communication notification 108 may be permitted to stay pending, and/or may be stored and re-communicated (e.g., according to operation 1130) when the sleep priority level drops and/or the user 101 ceases to have cognitive state monitored according to the sleep map 500.

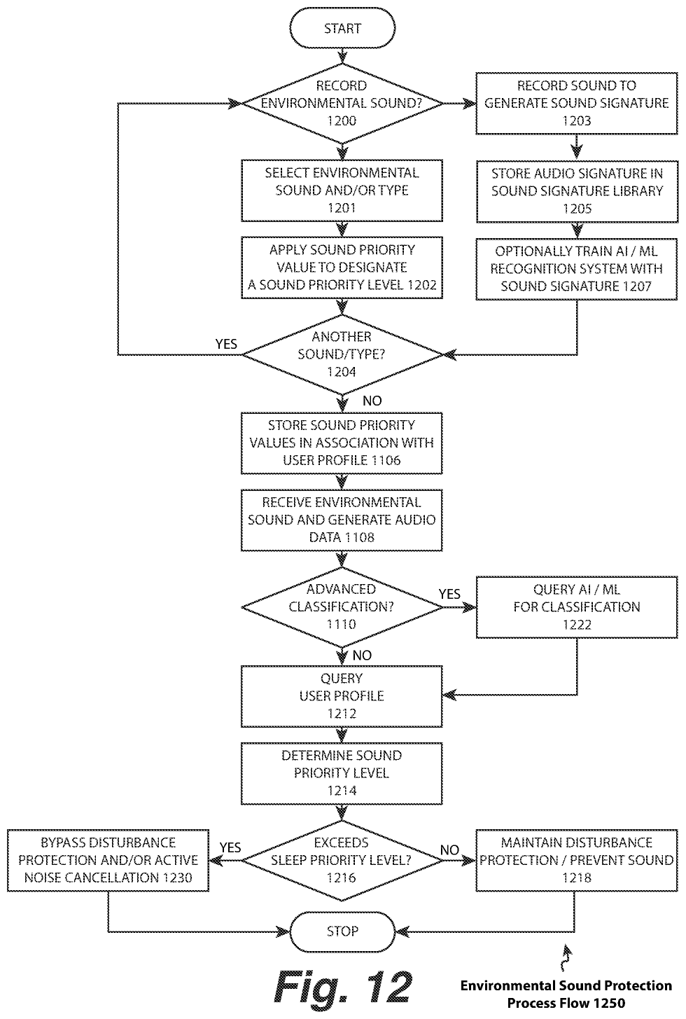

FIG. 12 illustrates an environmental sound protection process flow 1250, according to one or more embodiments. Operation 1200 may determine whether to record an environmental sound 104. The environmental sound 104 may be recorded in preparation for generation of a default sound signature library 261, and/or may be a user-defined recording by the user 101, according to one or more embodiments. Where an environmental sound 104 is to be recorded, operation 1200 may proceed to operation 1203 which may record the environmental sound 104 to generate a sound signature 262. Operation 1205 may then store the sound signature 262 in the sound signature library 161, and operation 1207 may optionally train an artificial intelligence system and/or machine learning system (e.g., the AI/ML system 244) to recognize the environmental sound 104 by utilizing the sound signature 262 or features thereof as training data. Operation 1207 may then proceed to operation 1202.

Where no environmental sound 104 is to be recorded, operation 1200 may proceed to operation 1201, which may select an environmental sound 104 and/or type of sound (e.g., street noise, distant siren, wall or ceiling banging, etc.) that may be preexisting in a sound signature library 261 or previously recorded. Operation 1202 may then apply a sound priority value to designate a sound priority level, the application either to the selected environmental sound, type of environmental sound 104, and/or the recorded environmental sound 104, according to one or more embodiments.

Operation 1204 determines whether another sound, type, and/or recording should be defined and/or have an associated sound priority level, in which case operation 1204 may return to operation 1200. If no additional sound priority levels are to be defined, operation 1204 may proceed to operation 1106. It should be noted that one or more devices utilizing the process flow of FIG. 12 may do so to overwrite default settings for environmental sounds 104. For example, the sound signature library 261 may include default values that a user 101 may override, for example by following a UI workflow implementing one or more processes of the environmental sound protection process flow 1250.

In one or more embodiments, sound signature libraries 261 may be prepared for a given environment, both to generate noise canceling, generate masking wave forms, and/or define high priority disturbance events 102. In one or more embodiments, the user 101 may also engage in one or more processes in realtime and during an attempted sleep period. For example, the user 101 may input a gestural input that initiates recording in operation 1203 (e.g., on the microphone 209, the microphone 312, the microphone 409). This may help immediately create a sound signature 262 and/or create a masking waveform to define a low priority disturbance event 102 and/or create masking waveforms, respectively. Operation 1106 may store one or more sound priority values in association with a user profile 252. Alternatively, where device running one or more processes of FIG. 12 is only intended for one user 101, the one or more sound priority values may be stored in a different location on a computer readable memory, for example in association with a sound signature library 261.

Operation 1108 then receives an environmental sound 104 and generates an audio data that may include a waveform of the environmental sound 104. The environmental sound 104 may be received on a microphone (e.g., on the microphone 209, the microphone 312, and/or the microphone 409). Operation 1110 determines whether an advanced classification should be applied, in which case operation 1110 proceeds to operation 1222 which may query an artificial intelligence and/or machine learning system (e.g., the AI/ML system 244) for classification. Operation 1222 may then proceed to operation 1212. Operation 1110 may also advance to operation 1212 if no advanced classification is to occur.

Operation 1212 queries a user profile 252 and/or another location to determine a sleep priority level. Operation 1214 may then determine a determine the sound priority level, either through matching the environmental sound 104 to the sound signature 262 through audio and/or waveform analysis, and/or through the AI/ML classification. Operation 1214 may then proced to operation 1216.

Operation 1216 determines whether the sound priority level exceeds the sleep priority value, in which case operation 1216 proceeds to operation 1230 which may bypass disturbance protection and/or bypass active noise cancelation of a speaker such as integration in a set of earphones 300. Where the sound priority level does not exceed the sleep priority level, operation 1216 may proceed to operation 1218 that may maintain disturbance protection and/or prevent the environmental sound 104 from reaching and/or perceptibly manifesting for the user 101 (e.g., maintain active noise canceling, generating and maintaining canceling waveforms and/or masking sounds, sending a silencing command 266, etc.).

It should be noted that, in both FIG. 11 and FIG. 12, operation 1112 and operation 1212 are not necessary, according to one or more embodiments. For example, in one or more embodiments all disturbance events 102 classified as “high priority” may be allowed to disturb the user 101, and all disturbance events 102 classified as “low priority” may not be allowed to disturb the user 101.

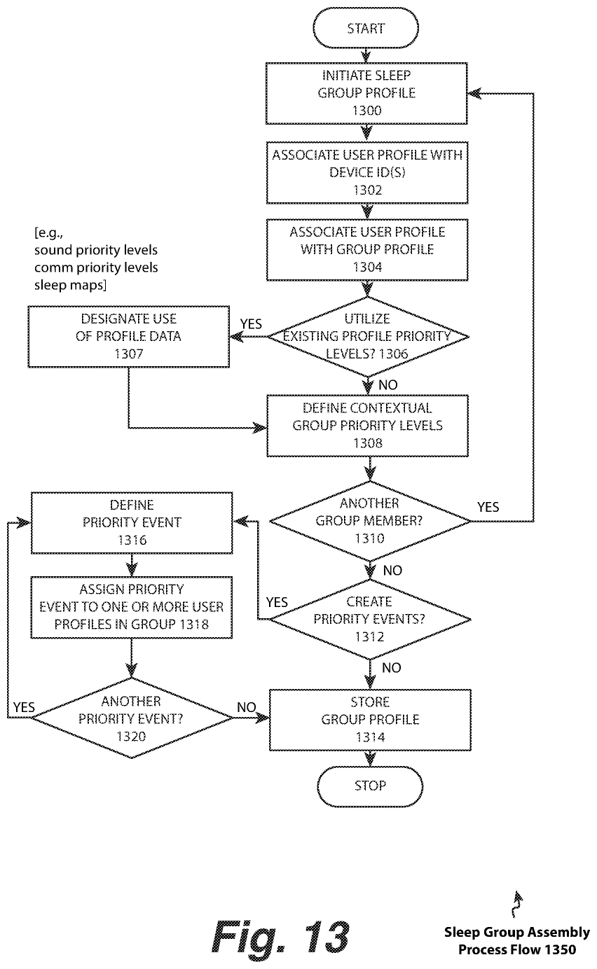

FIG. 13 illustrates a sleep group assembly process flow 1350, according to one or more embodiments. Operation 1300 initiates a sleep group profile (e.g., the sleep group profile 272). The sleep group profile 272 may be assigned a unique identifier, e.g., the group UID 273. Operation 1302 associates a user profile (e.g., the user profile 252) with one or more devices, for example the earphones 300 and/or one or more communication devices 400. In one or more embodiments, the devices may be associated within a list of devices, which may be identified through a device UID 255 (e.g., a device identifier, a MAC address, a static network address associated with the device, etc.). Operation 1304 associates the user profile 252 with the sleep group profile 272. For example, operation 1304 may add a user UID 253 to a set of user UIDs 253 within the sleep group profile 272. Alternatively or in addition, no user profiles 252 are utilized, and device UIDs 255 may be directly stored as “users” or “members” of the sleep group profile 272.

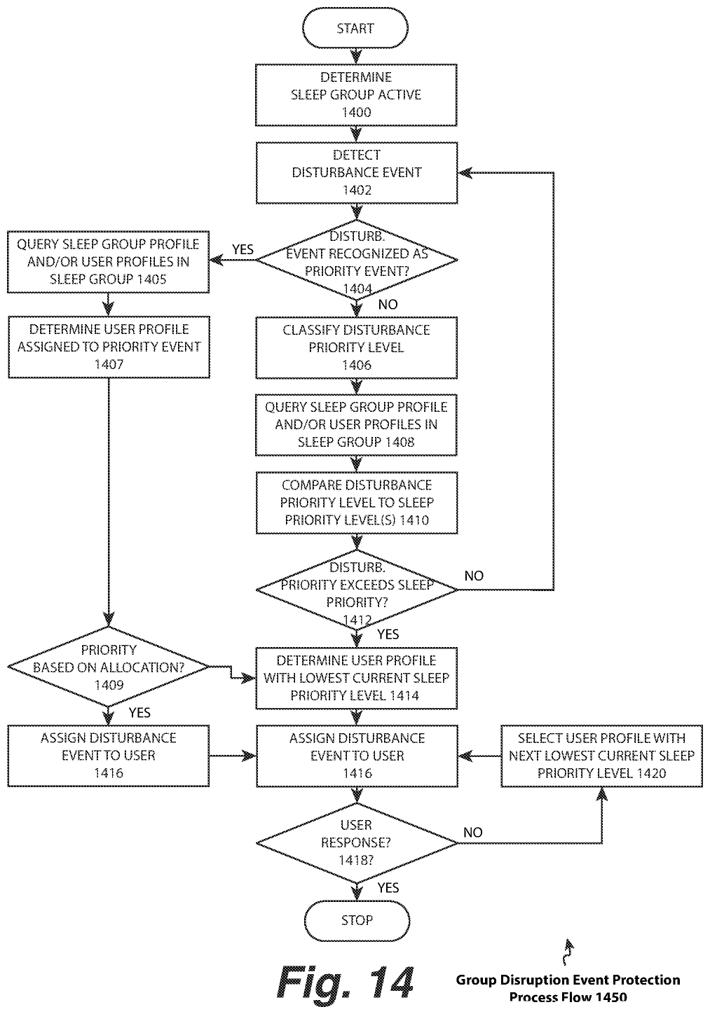

Operation 1306 determines whether to utilize an existing profile priority level, for example either a sleep priority level (as may be defined in a sleep map 500 or elseware) and/or disturbance event 102 priority level (e.g., a sound priority level, a communication priority level). Where existing data should be utilized, operation 1306 proceeds to operation 1307 which may designate use of profile data for the user profile 252 of the user 101, for example granting access to sleep maps 500, priority instances of the event data 256, and/or other data.

Where new priority levels are to be defined, which may be limited in context to when that sleep group 601 is active, operation 1306 may proceed to operation 1308. Similarly, even where profile data is utilized to establish baselines and/or default priority values, operation 1307 may proceed to 1308 which may define additional and/or overriding priority levels. Operation 1308, therefore, may establish contextual group priority levels that occur when the sleep group 601 is functioning and/or in effect. The sleep group 601 may be determined to be in effect in a number of circumstances, as further shown and described in conjunction with the embodiment of FIG. 14.

Operation 1310 determines whether another group member should be added to the sleep group 601 (e.g., another user 101 and/or an associated device or user profile 252 should be added to the sleep group profile 272), in which case operation 1310 returns to operation 1300.

In one or more embodiments, it will also be recognized that different group profiles 272 may be active at the same time and/or group profiles 272 may automatically come into or out of use depending on context. For example, in a household where two spouses both generally sleep at the same time, and also an elderly family member sometimes visits for several nights in a row, a first group profile 272A may be defined for the spouses, whereas a second group profile 272B may be defined for the spouses plus the elderly family member. In such case, each of the first group profile 272A and the second group profile 272B have different rules and may automatically be switched when the earphones 300 or another indicator of the elderly family member is detected. If no additional members are to be added, operation 1310 may proceed to operation 1312.

Operation 1312 may define one or more priority events (e.g., the priority instances of the event data 256). If one or more priority instances of the event data 256 are to be defined, operation 1312 may proceed to operation 1316, and otherwise to operation 1318. Operation 1316 may define a priority event that may be a set of data attributes and/or values that define a disturbance event 102 or other event that, upon occurrence, should be directed to and/or routed to one or more users 101, for example by notifying the user 101 through the earphones 300 of the user 101 and/or another device of the user 101. Operation 1318 may then assign the priority event to a user profile 252 within the sleep group 601. Operation 1318 may also assign the priority event to one or more other user profiles 252, for example as contingency recipients should there be no response from the primary user profile 252 to which the priority event is assigned. Alternatively, or in addition, the priority event may specify two or more user profiles 252, where the user profile 252 to which the priority event is routed may depend on availability or cognitive state of each user 101, and/or the sleep priority values associated with the user 101. For instance, where two users 101 (e.g., a user 101.1 and a user 101.2) each have a sleep map defined (e.g., a sleep map 500.1 and a sleep map 500.2), a disturbance event 102 may be routed to the user 101.2 if the user 101.1 is in a sleep period having a sleep priority value of ‘4’ and the user 101.2 is in a sleep period having a sleep priority value of ‘3’. Operation 1320 determines whether another priority event should be defined, in which case operation 1320 may return to operation 1316. If no additional priority event is to be defined, operation 1320 proceeds to operation 1314, which may store the sleep group profile 272 (e.g., in a database such as the database 270).

FIG. 14 illustrates a group disturbance event protection process flow 1450, according to one or more embodiments. Operation 1400 determines a sleep group (e.g., the sleep group 601) is active. The sleep group 601 may be determined to be in effect in a number of circumstances, for example where two or more sleep maps 500 are actively in use, when two or more sleep session data 260 are being recorded, where two or more earphones 300 or other devices are communicatively coupled to the same instance of the coordination hub 200 (e.g., the coordination hub 200 may be owned by a commercial company with employees bringing their own earphones 300), etc. Operation 1402 may detect a disturbance event.

Operation 1404 may determine whether the disturbance event 102 is recognizable as a priority instance of the event data 256. In one or more embodiments, a disturbance event 102 may be classified not only according to priority level, but also if the disturbance event 102 matches criteria of the priority instance of the event data 256, as shown and described throughout the present embodiments. Where the disturbance event 102 is recognized as the priority event, operation 1404 may proceed to operation 1405 which may query the sleep group profile 272 and/or user profiles 252 within the sleep group profile 272, and operation 1407 may then determine one or more user profiles 252 assigned to the priority instance of the event data 256 (e.g., a subgroup of the sleep group 601) before proceeding to operation 1409. Operation 1409 may determine whether the priority instance of the event data 256 should be assigned to the associated subgroup of the sleep group 601, in which case operation 1409 proceeds to operation 1414. Where no priority based allocation is to be utilized, operation 1409 may proceed to operation 1411 which may determine a user hierarchy for assignment (which may be skipped where only a single user 101 is within the subgroup), then proceed to operation 1416.

Returning discussion to operation 1404, where the disturbance event 102 is not recognized as a priority instance of the event data 256, operation 1404 may proceed to operation 1406, which may classify a disturbance priority level (e.g., a sound priority level, a communication priority level), for example through one or more devices, systems, and/or methods shown and described throughout the present embodiments.

Operation 1408 may query the sleep group profile 272 and/or one or more user profiles 252 within the sleep group profile 272 to determine sleep priority levels (and/or cognitive states) of each of the user 101 associated with the user profiles 252. For example, each user 101 within the sleep group 601 that is having cognitive state actively tracked and/or is otherwise communicatively coupled to a coordination hub 200 may have their sleep map 500 queried to determine sleep priority. Alternatively, or in addition, the sleep session data 260 of each may be queried to determine present cognitive states (e.g., a sleep state, an awake state, a REM state). Operation 1410 then compares the disturbance priority level to the sleep priority level of each of the user profiles 252.

Operation 1412 determines whether the disturbance priority level exceeds the sleep priority level. If not, the disturbance event is discarded (or data describing the disturbance event 102 may be stored for later delivery), and operation 1412 may return to operation 1402. Where the disturbance event 102 does exceed the sleep priority level of at least one member of the sleep group 601, operation 1412 may proceed to operation 1414 which may determine a user profile 252 with a lowest current sleep priority level, for example as determined from an active sleep map 500 and/or sleep session data 260.

Operation 1418 optionally may determine if a user 101 responds to the disturbance event 102 once the disturbance event 102 is permitted to perceptibly manifest for the user 101, for example by passing through audio of a communication notification 108 and/or turning of noise canceling capabilities of the earphones 300. The user 101 response may be determined through directly detecting a user response (e.g., picking up a phone call, making a sound in response to an environmental sound 104) and/or through an inferred response (e.g., the user 101 entering a sustained macro movement period and/or changing cognitive state to an awake state). If the user 101 does not respond, operation 1418 may proceed to operation 1420 which may select a different user 101 associated with a different user profile 252, for example the next lowest current sleep priority level. The new user 101 may then be re-assigned the disturbance event 102 in operation 146. If the user 101 does respond in operation 1418, operation 1418 may then proceed to end.

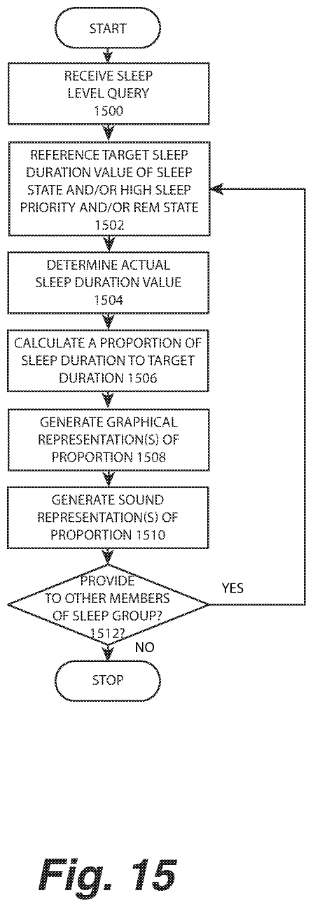

FIG. 15 illustrates a low cognitive load sleep query process flow 1550, according to one or more embodiments. Operation 1500 receives a sleep level query from a user 101. The user 101 may initiate the query through a variety of means, for example speaking and/or whispering into the microphone 312 of the earphones 300, providing a gesture on the touch interface 305, pressing a button on the coordination hub 200, sitting up in bed as detected from an accelerometer, etc.

Operation 1502 may reference a target sleep duration value. The target sleep duration value may be a target for all sleep states, for a high priority sleep state, and/or for a REM sleep state. The target may be specified and stored as data in one or more locations, including without limitation the user profile 252 and/or the sleep map 500 that may be in active use. Operation 1504 may then determine the actual sleep duration value, including for all sleep states, high priority sleep states, and/or for a REM sleep states, as the case may be. Operation 1506 calculates a proportion of the sleep duration to the target duration, which will generally be a number between 0% and 100%. However, in one or more embodiments, it will be noted that the user 101 could sleep more than 100% of a target value, and one or more of the below representations may include graphical or sound elements to represent such over-sleep. In one or more other embodiments, two or more sleep levels may be calculated, for example one proportion for total sleep, and one proportion for REM sleep. In one or more other embodiments, sleep score proportion based on aggregate weighted values can be utilized.

Operation 1508 generates a graphical representation of the proportion. For example, the graphical representation may be a numerical percentage, a numerical fraction, an analogy for the proportion (e.g., a filled battery, a color), etc. The graphical representation may be rendered on an indicator light on a device (e.g., the coordination hub 200), an illumination light of a device (e.g., a light lighting up the earphones within a case for the earphones 300), on the communication device 400, and/or else ware.

Operation 1510 generates a sound representation of the proportion. For example, the sound representation may be one or more tones, chimes, musical soundbites, ambient music, sound analogy (e.g., a full battery sound common to electronics), spoken words (e.g., ‘need rest’, or ‘sleep sufficient’), etc. The sound may be generates on the speaker 308 of the earphones 300, the speaker 408 of the communication device 400, a speaker of the 208 of the coordination hub 200, and/or a different speaker).

Should a sleep group 601 have been specified and is active, operation 1512 determines if the sleep level query should also display sleep levels of one or more other members of a sleep group 601. In the case other sleep group 601 members (e.g., the users 101.1, the user 101.2, etc.) should have their sleep levels shared with the querying user 101, operation 1512 may return to operation 1502 where the other sleep levels for display may be queried and calculated. Sleep levels of each user 101 may be presented on a display in sequence (e.g., first display a first graphical representation of the sleep level of a first user 101.1 and then display a second graphical representation of a sleep level of a second user 101.2) or simultaneously (two or more sleep levels of two or more users 101 contemporaneously displayed on a display, such that sleep data of the entire sleep group 601 may be visible to the querying user 101).

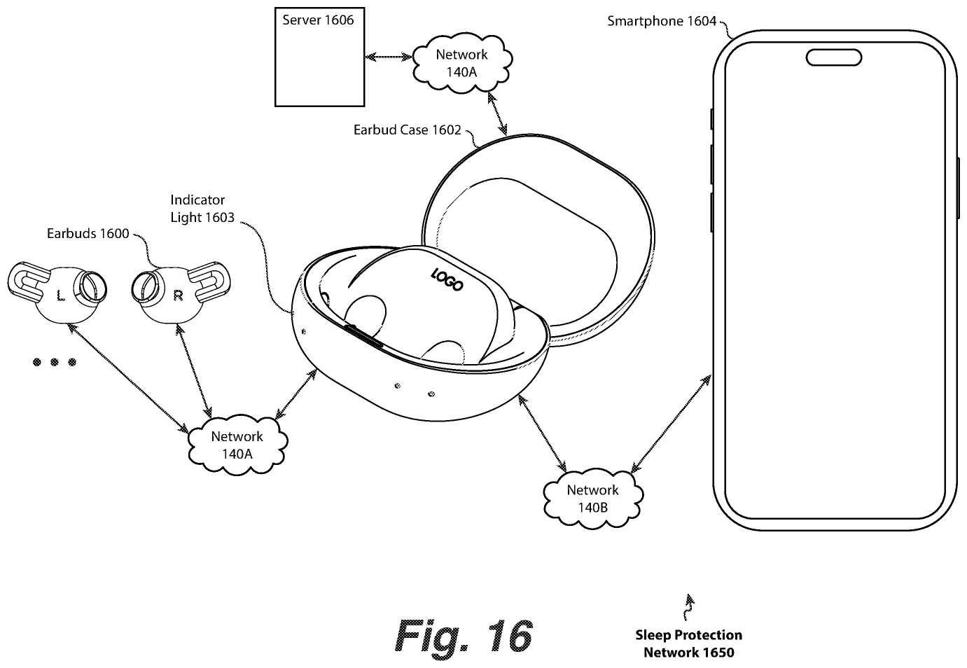

FIG. 16 illustrates an example of the sleep protection network 150, utilizing a set of earbuds 1600 implementing the earphones 300, an earbuds case 1602 also implementing the coordination hub 200, and a smartphone 1604 implementing the communication device 400, the sleep protection network 150 of FIG. 16 referred to as the sleep protection network 1650, according to one or more embodiments. The earbuds case 1602 may include an internal chargeable battery that may be charged through a USB-C connection to a DC power supply. The earbuds 1600 may be placed into form-fitting recesses in the earbud case 1602 and/or may be held in place through magnetic connections, where charging contacts on the earbuds 1600 and the earbud case 1602 may meet to transfer power from the earbud case 1602 to each of the earbuds 1600. The earbud case 1602 may be initially configured and/or controlled by an application running on the smartphone 1604 (e.g., an iPhone 14®, an Android® device). However, in the present embodiment, the earbud case 1602 may be able to operate independently of the smartphone 1604 such that the user 101 may have minimal sleep distraction. The earbuds 1600 may be communicatively coupled the earbud case 1602 through wireless network interface controllers supporting a Bluetooth® and/or a low energy protocol (e.g., the low energy protocol 315, such as a low-power Bluetooth® connection), for example forming the network 140A. The earbud case 1602 may be similarly communicatively coupled to the smartphone 1604 through wireless network interface controllers supporting a Bluetooth® and/or low-power Bluetooth® connection, and/or through wireless network interface controllers supporting WiFi and/or cellular (e.g., LTE, 5G connection), for example forming the network 140B. In the present example, the earbud case 1602 may be connected to WiFi and/or another wireless protocol such that it can access any supporting processes or servers (e.g., the server 1606), for example to make remote procedure calls (RPCs), remotely store or backup user profiles or data, and/or download or stream music, etc. This may further assist in ensuring that the user 101 can be autonomous and/or separated from their communication device 400 such as the smartphone 1604.

In the example embodiment of FIG. 16, a user 101 may be suffering from poor sleep quality and/or wish to improve their sleep quality. There may be multiple potential disturbances that the user 101 may experiences during both nightly sleep and naps. In the present example, the user 101 has a job that sometimes requires unusual work hours (e.g., needing to communicate with an international team of employees), is a new parent, and/or must often sleep in an environment (e.g., the sleep environment 100) with potentially disruptive environmental sounds 104 (e.g., a crying newborn, outside street traffic, and/or a partner snoring).

The user 101 may first configure the sleep protection network 1650, for example through a companion app download from the Apple® App Store or Google® Play Store. The user 101 may then connect the earbud case 1602 to the smartphone 1604, for example through Bluetooth® pairing, joining the same or similar WiFi networks, and/or through other methods. Once communicating, the user 101 may be able to set up and configure the earbud case 1602 and/or the earbuds 1600, including setting up WiFi or other network connections enabling less or no reliance of the smartphone 1604 (e.g., the network 140C).

The user 101 may first choose or download one or more sleep tracks (e.g., the audio track 320), which may be played for the user 101 to help the user fall asleep and/or back to sleep. The audio tracks 320 may be stored and/or streamed from the smartphone 1604, may be stored on or streamed from the earbud case 1602, and/or may be streamed from a remote server (e.g., the server 1606) through to and/or through the earbud case 1602. The earbuds 1600 may include one or more UI/UX interactions to select or switch between sleep tracks, noise canceling modes, and/or noise masking modes (e.g., the touch interface 305), and/or one or more buttons or other touch elements of the earbud case 1602 to do the same.

The user 101 may then select and/or define sleep periods, disturbance events 102, and their priorities. Some of the disturbance events 102 the user 101 may choose to have a low priority, and others a high priority. First, the user 101 may be able to select or specify a pattern of sleep that may fit different contextual needs of the user 101. As a more specific example, the user 101 may define a first sleep map (e.g., the sleep map 500X) intended for use when the user 101 is not “on-call” for work-related matters, a second sleep map (e.g., a sleep map 500Y) intended for use when the user 101 is “on-call” for work-related matters, and a third sleep map (e.g., a sleep map 500Z) intended for when the user 101 wishes to attempt an opportunist nap in a short period of calm between work and/or parenting responsibilities.

When setting up each sleep map 500, the user 101 may specify which sleep pattern is to be followed, and its relative priority. For example, for the sleep map 500X, the user 101 may simply have specified that the user 101 would like eight hours of undisturbed sleep (e.g., a high priority sleep period 504), initiated by detection of the user 101 falling asleep (e.g., as set by a state designator 511 for “sleep”). The user 101 may also define that a baby crying. as may be detected through an sound signature recorded by a microphone 312 of the earbuds 1600 and/or a microphone 209 of the earbud case 1602, may be of a higher relative priority than a contiguous 8-hour sleep period. During use of the sleep map 500X. the user 101 may have all communications 106 of the smartphone 1604 blocked from being passed through to the user 101, except for what may be defined as an emergency call from a family member, e.g., three calls and/or at least one call and one text message received on the smartphone 1604 within a one-minute period. The earbud case 1602, as an instance of the coordination hub 200, may therefore act as a form of “sleep firewall” screening disturbance events 102, environmental sounds 104, communications 106, and/or communication notifications 108.

The sleep map 500Y may be set up such that the user 101 receives at least one period of REM sleep lasting at least three hours in length, and at most five hours in length (e.g., a REM state 518), where until the user 101 has completed the REM state period the sleep priority value will be “high” (e.g., a high priority sleep period 504), and thereafter “medium”. During this period the user 101 may define the sound of a crying infant to be of “low priority”. therefore choosing not to permit the sound of the crying infant be able to potentially wake the user 101. Rather, the user 101 may have selected a low priority being that a spouse may need to care for the infant during this portion of the user 101's workweek. Alternatively, or in addition, a detected sound of the infant may increase in priority over time, for example starting at ‘low’, increasing to ‘medium’ after 4 minutes, and increasing to ‘high’ after 7 minutes. The user 101 may then define any number of communications 106, types of communications 106 (e.g., a message from a particular user on Slack® workplace messaging app, a message with certain keywords, etc.), communication notifications 108, and associated priority levels.

Finally, the user 101 may define a sleep map 500Z for naps. The sleep map 500Z acting as a sleep profile for the nap may define a short (e.g., 20 minute) period of high priority sleep, initiated from the detection of a sleep cognitive state of the user 101, followed by a short period of low priority sleep (e.g., 40 minutes). The sleep map 500Z may allow for almost any communication 106 to have a high priority and almost any environmental sound 104 to have a medium priority. This can help ensure that the user 101 can obtain a short “cat nap” while still being responsive to important events.

Prior to sleeping, the user 101 may select the audio track 320 and the appropriate instance of the sleep map 500 and then place the earbuds 1600 in the ears of the user 101. The earbuds 1600 may include capabilities for active noise canceling, white noise, and/or masking sounds, including without limitation playing the audio track 320 as a masking track. The user 101 may therefore have environmental sounds 104 abated and be able to begin falling to sleep. The user 101 may be able to listen to and benefit from the white noise, masking sounds, and/or masking track (e.g., the audio track 320) without the need for connection between the smartphone 1604 and the earbud case 1602.

A set of sensors 310 on the earbuds 1600 may gather physiological data 110 from the user 101. In the present example, the earbuds 1600 may include an accelerometer 314 and a gyroscope 316 which may be utilized to gather data for detecting both small movements (e.g., heart beats, beaths) and large movements (e.g., the user 101 rolling over or sitting up). The physiological data 110 may be transmitted through the Bluetooth® connection (e.g., via the network 140A) to the earbud case 1602 for assessment. In a preferred embodiment, a low energy audio protocol may be used (e.g., the low energy protocol 315). Data analysis and/or processing may first determine individual indicators of heart beats and/or beaths (e.g., the indicator data 120), and then features, such as heart rate and respiration rate, extracted (e.g., the feature data 124). The features may be used to determine a cognitive state of the user 101, for example whether the user 101 is beginning to fall asleep (e.g., a pre-sleep state), has fallen asleep (e.g., a sleep state), and/or is in a rapid-eye movement phase (e.g., a REM state), as shown and described herein and/or as may be known in the art.

In the present example, the user 101 may chose to use the sleep protection network 1650 on a ‘worknight’ in which the user 101 must be ‘on call’. The user 101 may therefore select a sleep profile such as the sleep map 500X. The user 101 places the earbuds 1600 in their ears and active noise cancelation and/or a masking audio track 320 may be initiated. The physiological data 110 of the user 101 may be gathered, transmitted to the earbud case 1602, and then analyzed to determine the cognitive state of the user 101 over time periods and/or epochs (e.g., each 30 second periods). Once the user 101 is asleep, they may be determined to be in the “high value” sleep period by computing processes of the earbud case 1602, as shown and described herein. The user 101 may receive a first phone call (e.g., the user 101) that may be work-related, but it may only be determined to be of a ‘medium’ priority, and therefore the user 101 not notified and/or a communication notification 108 associated with the communication 106 not permitted to reach the user 101, for example by ensuring the environmental sound 104 of the smartphone 1604 remains actively canceled and/or masked, and that no sound representing the communication notification 108 is passed to the speakers 308 of the earbuds 1600.

The earbud case 1602 may track the length of the first REM state experienced by the user 101. For example, the user 101 may enter and maintain the REM state for a period of 3 hours and 50 minutes, after which the user 101 may enter a NREM state. Upon entering the NREM state, the sleep priority level may drop to a ‘medium’ level. Operationally, any ‘medium’ level communications 106 may be delivered to the user 101 and/or associated communication notifications 108 passed to the user 101 as soon as the user 101 enters the NREM state.

In the present example, a high priority disturbance event 102 is permitted to wake the user 101. For example, an environmental sound 104, as may be gathered by a microphone of the earbuds 1600 and/or the earbud case 1602 (e.g., the microphone 312 and/or the microphone 209, respectively), may be determined to be a crying baby following audio analysis (e.g., matching to a sound signature of the sound signature library 261). Where the crying persists past 7 minutes, the priority may be raised to ‘very high’. For example, the spouse of the user 101 may be working in a different room or may be taking a shower and therefore may not hear the crying infant. The user 101 may be slowly woken up, for example but reducing noise canceling and/or masking, by playing a notification sound in the speakers of the earbuds 1600, by actively playing the environmental sound 104 on the speaker 308 of the earbuds 1600, etc. As a result, the user 101 may enter the awake state such that they may respond. Wakefulness may be detected through accelerometer and/or gyroscope data to terminate the alarm and/or determine the user 101 is in the awake state. Following resolution of activity causing the disturbance event 102, the sleep map 500 may re-initiate, either at the beginning or where it was paused or terminated. For example, if the user 101 only had achieved one hour of REM sleep when subject to the disturbance event 102, the timer may resent to require another undisturbed period of 3 to 5 hours of REM sleep, or may only require 2 to 4 hours of REM sleep (the original period minus one hour of REM sleep already achieved).

Often, the user 101 may wish to know what time it is in the middle of a sleep period. However, more commonly, the user 101 may actually wish to know how much sleep they have achieved. However, running a calculation of the amount of time the user 101 has slept based on the clock, and/or letting the bright light of the clock enter the retina, may cause the user 101 in increase in wakefulness, further inhibiting sleep. In the present example, the earbud case 1602 may include one or more dim but visible indicator lights, for example illuminating the interior of the earbud case 1602, set in the exterior of the housing of the earbud case 1602, and/or in other locations. The one or more indicator lights may be used to communicate a sleep level and/or amount of sleep the user 101 has achieved. For example, five out of eight indicator lights being lit may indicate the user 101 has achieved five out of eight hours of sleep. In another example, a green indicator light may indicate the user 101 has achieved at least one full REM sleep period and at least six hours of total sleep. The indicator lights may also remain off unless the sleep level is queried by the user 101, for example by the user activating a touch interface 305 on the earbuds 1600 and/or tapping or gently moving the earbud case 1602.

As a result of the use of the earbuds 1600, earbud case 1602, and/or sleep protection network 1650, the user 101 may have increased confidence that they will be able to achieve necessary sleep while not missing important and/or emergency events. This piece of mind may be critical in helping the user 101 fall asleep more quickly, fall back asleep after any disturbance, achieve more rested sleep, and/or be able to sleep more often (e.g., utilizing naps) when they otherwise would not believe they could do so without missing important events.

It should be noted that, in the present example, multiple devices (e.g., a smartphone 1604A that may be a personal phone, a smartphone 1604B that may be a work phone) may be simultaneously paired with the earbud case 1602, and any disturbance events 102 therefrom individually and collectively managed, screened, and selectively passed to the user 101.

It will be appreciated that one or more of the systems, routines, subroutines, modules, and other functions and elements of the coordination hub 200 may be performed by the earphones 300, including miniaturized earbuds. For example, increasingly powerful electronics may allow one or more of the present embodiments to be entirely implemented within the earphones 300 at commercially reasonable production cost, and this disclosure will be recognized able to encompass such advances in general technology allowing for further miniaturization of data processing and storage devices.

In one or more embodiments, the coordination hub 200 may generate a message and/or alert for a first user 101.1 when a second user 101.2 wakes, changes cognitive state (e.g., leaves an REM state), achieves a sufficient aggregate sleep score, etc. They may ensure that users 101 who may have something urgent, or need to change shifts, can find the most opportune time to do so to maximize sleep of the second user 101.2. The message may be sent to the communication device 400 of the user 101.1.

In one or more embodiments, the length of time over which a disturbance event 102 occurs may change its priority as classified. For example, a baby crying may increase in its priority level, linearly, exponentially, or according to another function, until it overcomes the sleep priority level of the user 101.

It will be appreciated that one or more of the systems, routines, subroutines, modules, and other functions and elements of the coordination hub 200 may be performed by the earphones 300, including miniaturized earbuds. For example, increasingly powerful electronics may prevent one or more of the present embodiments to be entirely implemented within the earphones 300, and this disclosure will be recognized able to encompass such advances in general technology allowing for further miniaturization of data processing and storage devices.

Although the present embodiments have been described with reference to specific example embodiments, it will be evident that various modifications and changes may be made to these embodiments without departing from the broader spirit and scope of the various embodiments. For example, the various devices, engines, agent, routines, and modules described herein may be enabled and operated using hardware circuitry (e.g., CMOS based logic circuitry), firmware, software, or any combination of hardware, firmware, and software (e.g., embodied in a non-transitory machine-readable medium). For example, the various electrical structure and methods may be embodied using transistors, logic gates, and electrical circuits (e.g., application specific integrated circuitry (ASIC) and/or Digital Signal Processor (DSP) circuitry).

In addition, it will be appreciated that the various operations, processes, and methods disclosed herein may be embodied in a non-transitory machine-readable medium and/or a machine-accessible medium compatible with a data processing system (e.g., the coordination hub 200, the earphones 300, the communication device 400, one or more servers communicating with any of the foregoing including as may run the AI/ML system 244, etc.). Accordingly, the specification and drawings are to be regarded in an illustrative rather than a restrictive sense.

The structures in the figures such as the engines, routines, and modules may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the preceding disclosure.

Embodiments of the invention are discussed above with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures.

From reading the present disclosure, other variations and modifications will be apparent to persons skilled in the art. Such variations and modifications may involve equivalent and other features which are already known in the art, and which may be used instead of or in addition to features already described herein.

Although claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any claim and whether or not it mitigates any or all of the same technical problems.

Features which are described in the context of separate embodiments may also be provided in combination in a single embodiment. Conversely, various features which are, for brevity, described in the context of a single embodiment, may also be provided separately or in any suitable sub-combination. The applicants hereby give notice that new claims may be formulated to such features and/or combinations of such features during the prosecution of the present application or of any further application derived therefrom.

References to “one embodiment,” “an embodiment,” “example embodiment,” “various embodiments,” “one or more embodiments,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least one or more embodiments of the invention” includes the stated particular feature, structure, or characteristic.

The enumerated listing of items does not imply that any or all of the items are mutually exclusive, unless expressly specified otherwise.

It is understood that the use of a specific component, device and/or parameter names are for example only and not meant to imply any limitations on the invention. The invention may thus be implemented with different nomenclature and/or terminology utilized to describe the mechanisms, units, structures, components, devices, parameters and/or elements herein, without limitation. Each term utilized herein is to be given its broadest interpretation given the context in which that term is utilized.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; a smartphone, application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, one or more embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java.™, Jini.™, C, C++, Smalltalk, Perl, UNIX Shell, Visual Basic or Visual Basic Script, Virtual Reality Markup Language (VRML), ColdFusion.™. or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The term “processor” may refer to any device or portion of a device that processes electronic data from registers and/or memory to transform that electronic data into other electronic data that may be stored in registers and/or memory. A “computing platform” may comprise one or more processors.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing the sleep protection network 150 and devices and elements thereof according to the present invention will be apparent to those skilled in the art. Various aspects of the invention have been described above by way of illustration, and the specific embodiments disclosed are not intended to limit the invention to the particular forms disclosed. The particular implementation of the the sleep protection network 150 and devices and elements thereof may vary depending upon the particular context or application. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A device for enhancing a sleep a user, the device comprising:
a housing,
a processor,
a network interface controller configured to communicatively couple the device to an earphone of the user and at least one of a server and a communication device, and
a memory comprising:
an indicator identification subroutine comprising computer readable instructions that when executed:
extract an indicator data from a physiological data received from one or more sensors of the earphone of the user, the indicator data indicating at least one of a respiration of the user, a heartbeat of the user, and a macro movement of the user;
a feature extraction routine comprising computer readable instructions that when executed:
determine a physiological feature of the user comprising at least one of a respiration rate of the user, a heart rate of the user, a heart rate variability of the user, and a macro movement period of the user, and
assemble a feature data comprising the physiological feature;
a cognitive state determination routine comprising computer readable instructions that when executed:
determine a cognitive state of the user based on the physiological feature, and
record the cognitive state in association with a sleep session data;
a communications agent comprising computer readable instructions that when executed:
receive a communication notification that is at least one of a call notification, a message notification, and an app notification;
a sleep protection routine comprising computer readable instructions that when executed:
query the sleep session data,
determine that the user is in at least one of a sleep state and a pre-sleep state, and
deny passthrough of the communication notification to a speaker of the earphone to protect the sleep of the user.

2. The device of claim 1, wherein the memory further comprising:
a sleep structuring engine comprising computer readable instructions that when executed:
initiate a sleep priority map,
apply a first sleep priority value to a first period of the sleep priority map to designate a high priority period,
wherein the first period is at least one of (i) a user defined period and (ii) a REM period,
apply a second sleep priority value to a second period of the sleep priority map to designate a low priority period,
wherein the second period is at least one of (i) a different user defined time period; and (ii) an NREM period;
a communication classification engine comprising computer readable instructions that when executed:
classify the communication notification as a low priority communication; and
wherein the sleep protection engine further comprising computer readable instructions that when executed:
query the sleep session data,
determine that the user is in the high priority period, and
prevent a perceptible manifestation of the communication notification to protect a high priority sleep period of the user.

3. The device of claim 1, wherein the memory further comprising:
an audio collection agent comprising computer readable instructions that when executed:
receive an environmental sound from the environment of the user collected on at least one of a microphone of the device, a microphone of the communication device, and a microphone of the earphone, and
generate an audio data from the environmental sound;
a sound classification engine comprising computer readable instructions that when executed:
compare the audio data to a sound signature in a sound signature library, wherein the sound signature was collected and added to the sound signature library by the user, and
classify the audio data as a high priority sound;
wherein the sleep protection engine further comprising computer readable instructions that when executed:
determine a sound priority level of the audio data exceeds a sleep priority level, and
bypass an active noise canceling of the earphone to at least one of allow passthrough of the environmental sound and recreate the environmental sound on the speaker of the earphone, to assist in initiating the sleep of the user by reassuring the user that important sounds will interrupt a high priority sleep state.

4. The device of claim 1, wherein the user is a first user and the earphone is a first earphone of the first user and the device configured for communicative coupling to a second earphone of a second user simultaneously with the first earphone of the first user, and the memory further comprising:
a sleep grouping subroutine comprising computer readable instructions that when executed:
associate a device ID of the first earphone with a user profile of the first user,
associate a device ID of a second earphone with a user profile of a second user, and
designate a sleep group profile comprising the user profile of the first user and the user profile of the second user;
an event designation subroutine comprising computer readable instructions that when executed:
define an event data, and
assign the event data to the user profile of the second user;
a disturbance classification engine comprising computer readable instructions that when executed:
receive a disturbance event, and
determine the disturbance event is defined in the event data;
a group allocation subroutine comprising computer readable instructions that when executed:
query at least one of the sleep group profile, the user profile of the first user, and the user profile of the second user to determine the second user is associated with the event data that is associated with the second user profile; and
wherein the sleep protection engine further comprising computer readable instructions that when executed:
generate an audio indicator of the event data on the earphone of the second user to protect the sleep state of the first user.

5. The device of claim 1,
wherein the sleep structuring engine further comprising computer readable instructions that when executed:
receive a sleep priority value designating a sleep priority level of at least one of a sleep period and a sleep cycle of the user;
wherein the communication classification engine further comprising computer readable instructions that when executed:
extract a text data from a communication associated with the communication notification,
query an artificial neural network, and
determine a communication priority level of the communication based on the text data; and
wherein the sleep protection engine further comprising computer readable instructions that when executed:
determine the communication priority level of the communication exceeds the sleep priority level, and
transmit a sound data associated with the communication notification to the speaker of the earphone, to assist in initiating the sleep of the user by reassuring the user that only important communications will interrupt the at least one of the sleep period and the sleep cycle of the user.

6. The device of claim 1, wherein the device further comprising a display, and wherein the memory further comprising:
a sleep proportion subroutine comprising computer readable instructions that when executed:
reference a target sleep duration value that is at least one of a default duration value and a custom duration value of the user,
determine an actual sleep duration value based on the sleep session data, and
calculate a proportion that is the actual sleep duration value relative to the target sleep duration value; and
a sleep sufficiency subroutine comprising computer readable instructions that when executed:
generate a first graphical representation of the proportion of the actual sleep duration value relative to the target sleep duration value through at least a first graphical element representing an extent of the actual sleep duration value and a second graphical element representing the proportion, the first graphical representation to decrease a cognitive load of the user in interpreting a sufficiency of the sleep, and
wherein the first graphical element represents a container and the second graphical element represents a filled portion of the container.

7. The device of claim 6, wherein the memory further comprising:
a gesture agent comprising computer readable instructions that when executed:
receive a sleep level query generated by a gesture input on a touch interface of the earphone;
wherein the sleep sufficiency subroutine comprising computer readable instructions that when generated:
display a clock on the display,
generate a second graphical representation of the proportion of the actual sleep duration value relative to the target sleep duration value comprising a third graphical element representing the proportion with a color, the second graphical representation to decrease the cognitive load of the user in interpreting the sufficiency of the sleep,
generate a sound representation of the proportion of the actual sleep duration value relative to the target sleep duration value comprising at least one of (i) a tone representing the proportion and (ii) a word describing the proportion, the sound representation played on the speaker of the earphone,
wherein at least one of the physiological data and the indicator data received from the earphone at a rate of between 20 Hz and 700 Hz,
wherein the physiological data and the indicator data received on a low energy protocol, and
wherein the coordination hub is pairable with a device generating the communication notification is recognizable by the device as an earphone device through a pairing communication protocol.

8. A method for protecting a sleep of a user, the method comprising:
receiving a physiological data that comprising at least one of a gyroscope data, an accelerometer data, a temperature data of the user, and a sound of the user, and optionally receiving an ambient temperature of an environment of the user,
wherein the physiological data is received on one or more sensors of an earphone;
extracting an indicator data that indicates at least one of a respiration of the user, a heartbeat of the user, and a macro movement of the user,
wherein the physiological data transmitted to a coordination hub;
determining a physiological feature of the user comprising at least one of a respiration rate of the user, a heart rate of the user, a heart rate variability, and a macro movement period of the user;
assembling a feature data comprising the physiological feature;
determining a cognitive state of the user based on the physiological feature;
recording the cognitive state in association with a sleep session data;
receiving a communication notification that is at least one of a call notification, a message notification, and an app notification;
querying the sleep session data;
determining that the user is in at least one of a sleep state and a pre-sleep state; and
denying passthrough of the communication notification to a speaker of the earphone to protect the sleep of the user.

9. The method of claim 8, further comprising:
initiating a sleep priority map,
applying a first sleep priority value to a first period of the sleep priority map to designate a high priority period;
wherein the first period is at least one of (i) a user defined period and (ii) a REM period;
applying a second sleep priority value to a second period of the sleep priority map to designate a low priority period;
wherein the second period is at least one of (i) a different user defined time period; and (ii) an NREM period;
classifying the communication notification as a low priority communication;
determining that the user is in the high priority period; and
preventing a perceptible manifestation of the communication notification to protect a high priority sleep period of the user.

10. The method of claim 8, further comprising:
generating an audio data from an environmental sound collected on a microphone from the sleep environment of the user;
classifying the audio data,
wherein a classification compares the audio data to an sound signature in an sound signature library, and
wherein the sound signature was collected and added to the sound signature library by the user;
determining a sound priority level of the audio data exceeds a sleep priority level; and
bypassing an active noise canceling of the earphone to at least one of allow passthrough of the environmental sound and recreate the environmental sound on the speaker of the earphone, to assist in initiating the sleep of the user by reassuring the user that important sounds will interrupt a high priority sleep state.

11. The method of claim 8, wherein the user is a first user and the earphone is a first earphone of the first user, the method further comprising:
associating a device ID of the first earphone with a user profile of the first user and associating a device ID of a second earphone with a user profile of a second user;
designating a sleep group profile comprising the user profile of the first user and the user profile of the second user;
defining an event data;
assigning the event data to the user profile of the second user;
receiving a disturbance event;
determining the disturbance event is defined in the event data;
querying the user profile of the first user and the user profile of the second user to determine the second user is associated with the event data that is associated with the second user profile; and
generating an audio indicator of the event data on the earphone of the first user to protect the sleep state of the first user.

12. The method of claim 8, wherein the earphone is a first earphone of a first user, the method further comprising:
associating a device ID of the first earphone with a user profile of the first user and a device ID of the second earphone with a user profile of a second user;
designating a sleep group profile comprising the first user profile and the second user profile;
receiving a disturbance event;
classifying the disturbance event as an event data;
querying the sleep session data of the first user and a sleep session data of the second user;
determining that the first user is in a high priority sleep state;
optionally determining that the second user is in a low priority sleep state; and
generating an audio indicator of the event data on the earphone of the second user to protect the high priority sleep state of the first user.

13. The method of claim 8, further comprising:
receiving a sleep priority value designating a sleep priority level of at least one of a sleep period and a sleep cycle of the user,
extracting at least one of a text data from a communication associated with the communication notification and a metadata of the communication associated with the communication notification;
inputting the text data into a communication classification engine comprising at least one of an artificial neural network (ANN) and a machine learning system (MLS);
determining a communication priority level of the communication based on the text data from at least one of an output of the ANN and an inference of the MLS;
determining the communication priority level of the communication exceeds the sleep priority level; and
passing a sound data associated with the communication notification to the speaker of the earphone, to assist in initiating the sleep of the user by reassuring the user that only important communications will interrupt the at least one of the sleep period and the sleep cycle of the user.

14. The method of claim 8, further comprising:
referencing a target sleep duration value that is at least one of a default duration value and a custom duration value of the user;
determining an actual sleep duration value based on the sleep session data;
calculating a proportion that is the actual sleep duration value relative to the target sleep duration value; and
generating for the user a representation of the proportion of the actual sleep duration value relative to the target sleep duration value through,
wherein the representation comprising a first graphical representation of the proportion of the actual sleep duration value relative to the target sleep duration value,
wherein the first graphical representation comprising at least a first graphical element representing an extent of the target sleep duration value and a second graphical element representing the proportion, the first graphical representation to decrease a cognitive load of the user in interpreting a sufficiency of the sleep, and
wherein the first graphical element represents a container and the second graphical element represents a filled portion of the container.

15. The method of claim 14, further comprising:
receiving a gesture input on a touch interface of the earphone;
optionally displaying a clock on a display; and
generating for the user the representation of the proportion in response to receiving the gesture input on the touch interface of the earphone,
wherein the representation further comprising a second graphical representation of the proportion of the actual sleep duration value relative to the target sleep duration value comprising a third graphical element representing the proportion with at least one of a color, a graphical texture, and a display frequency, the second graphical representation to decrease the cognitive load of the user in interpreting the sufficiency of the sleep,
wherein the representation further comprising a sound representation of the proportion of the actual sleep duration value relative to the target sleep duration value comprising at least one of (i) a tone representing the proportion and (ii) a word describing the proportion, the sound representation played on the speaker of the earphone,
wherein at least one of the physiological data and the indicator data received from the earphone at a rate of between 20Hz and 700Hz,
wherein the physiological data and the indicator data received on a low energy protocol, and
wherein the coordination hub is pairable with a device generating the communication notification is recognizable by the device as an earphone device through a pairing communication protocol.

16. A system for managing a sleep of a user, the system comprising:
an earphone comprising:
a speaker of the earphone,
one or more sensors configured to gather a physiological data of a user, and
a network interface controller of the earphone;
a coordination hub comprising:
a housing of the coordination hub,
a processor of the coordination hub,
a memory of the coordination hub,
a network interface controller of the coordination hub, and
a memory of the coordination hub comprising:
an indicator identification subroutine comprising computer readable instructions that when executed:
extract an indicator data from a physiological data received from the one or more sensors of the earphone of the user, the indicator data indicating at least one of a respiration of the user, a heartbeat of the user, and a macro movement of the user,
wherein the physiological data is received from the earphone on a low energy protocol;
a feature extraction routine comprising computer readable instructions that when executed:
determine a physiological feature of the user comprising at least one of a respiration rate of the user, a heart rate of the user, a heart rate variability of the user, and a macro movement period of the user, and
assemble a feature data comprising the physiological feature;
a cognitive state determination routine comprising computer readable instructions that when executed:
determine a cognitive state of the user based on the physiological feature, and
record the cognitive state in association with a sleep session data;
a communications agent comprising computer readable instructions that when executed:
receive a communication notification that is at least one of a call notification, a message notification, and an app notification;
a sleep protection routine comprising computer readable instructions that when executed:
query the sleep session data,
determine that the user is in at least one of a sleep state and a pre-sleep state, and
deny passthrough of the communication notification to a speaker of the earphone to protect the sleep of the user,
wherein the network interface controller of the coordination hub configured to communicatively couple to the earphone; and
a network communicatively coupling the earphone to the coordination hub.

17. The system of claim 16, further comprising:
a communication device comprising:
a processor of the communication device,
a memory of the communication device, and
a network interface controller of the communication device,
wherein the network interface controller of the coordination hub further configured to communicatively couple to the communication device,
wherein at least one of the communication device and the coordination hub further comprising:
a sleep structuring engine comprising computer readable instructions that when executed:
initiate a sleep priority map,
apply a first sleep priority value to a first period of the sleep priority map to designate a high priority period,
wherein the first period is at least one of (i) a user defined period and (ii) a REM period,
apply a second sleep priority value to a second period of the sleep priority map to designate a low priority period,
wherein the second period is at least one of (i) a different user defined time period; and (ii) an NREM period;
a communication classification engine comprising computer readable instructions that when executed:
classify the communication notification as a low priority communication; and
wherein the sleep protection engine further comprising computer readable instructions that when executed:
query the sleep session data,
determine that the user is in the high priority period, and
prevent a perceptible manifestation of the communication notification to protect a high priority sleep period of the user.

18. The system of claim 16, wherein at least one of the memory the coordination hub and the memory of the communication device further comprising:
an audio collection agent comprising computer readable instructions that when executed:
receive an environmental sound from the environment of the user collected on at least one of a microphone of the device, a microphone of the communication device, and a microphone of the earphone, and
generate an audio data from the environmental sound;
a sound classification engine comprising computer readable instructions that when executed:
compare the audio data to an sound signature in an sound signature library,
wherein the sound signature was collected and added to the sound signature library by the user, and
classify the audio data as a high priority sound;
wherein the sleep protection engine further comprising computer readable instructions that when executed:
determine a sound priority level of the audio data exceeds a sleep priority level, and
bypass an active noise canceling of the earphone to at least one of allow passthrough of the environmental sound and recreate the environmental sound on the speaker of the earphone, to assist in initiating the sleep of the user by reassuring the user that important sounds will interrupt a high priority sleep state.

19. The system of claim 16, wherein the user is a first user and the earphone is a first earphone of a first user and the coordination hub configured for communicative coupling to a second earphone of a second user simultaneously with the first earphone of the first user, and wherein at least one of the memory the coordination hub and the memory of the communication device further comprising:
a sleep grouping subroutine comprising computer readable instructions that when executed:
associate a device ID of the first earphone with a user profile of the first user,
associate a device ID of a second earphone with a user profile of a second user, and
designate a sleep group profile comprising the user profile of the first user and the user profile of the second user;
an event designation subroutine comprising computer readable instructions that when executed:
define an event data, and
assign the event data to the user profile of the second user;
a disturbance classification engine comprising computer readable instructions that when executed:
receive a disturbance event, and
determine the disturbance event is defined in the event data;
a group allocation subroutine comprising computer readable instructions that when executed:
query at least one of the sleep group profile, the user profile of the first user, and the user profile of the second user to determine the second user is associated with the event data that is associated with the second user profile; and
wherein the sleep protection engine further comprising computer readable instructions that when executed:
generate an audio indicator of the event data on the earphone of the second user to protect the sleep state of the first user;
wherein the sleep structuring engine further comprising computer readable instructions that when executed:
receive a sleep priority value designating a sleep priority level of at least one of a sleep period and a sleep cycle of the user;
wherein the communication classification engine further comprising computer readable instructions that when executed:
extract a text data from a communication associated with the communication notification,
query an artificial neural network, and
determine a communication priority level of the communication based on the text data; and
wherein the sleep protection engine further comprising computer readable instructions that when executed:
determine the communication priority level of the communication exceeds the sleep priority level, and
transmit a sound data associated with the communication notification to the speaker of the earphone, to assist in initiating the sleep of the user by reassuring the user that only important communications will interrupt the at least one of the sleep period and the sleep cycle of the user.

20. The system of claim 1,
wherein the coordination hub further comprising a display;
wherein at least one of the memory the coordination hub and the memory of the communication device further comprising a sleep proportion subroutine comprising computer readable instructions that when executed:
reference a target sleep duration value that is at least one of a default duration value and a custom duration value of the user,
determine an actual sleep duration value based on the sleep session data, and
calculate a proportion that is the actual sleep duration value relative to the target sleep duration value; and
wherein at least one of the memory the coordination hub and the memory of the communication device further comprising a sleep sufficiency subroutine comprising computer readable instructions that when executed:
generate a first graphical representation of the proportion of the actual sleep duration value relative to the target sleep duration value through at least a first graphical element representing an extent of the actual sleep duration value and a second graphical element representing the proportion, the first graphical representation to decrease a cognitive load of the user in interpreting a sufficiency of the sleep, and
wherein the first graphical element represents a container and the second graphical element represents a filled portion of the container;
wherein at least one of the memory the coordination hub and the memory of the communication device further comprising a gesture agent comprising computer readable instructions that when executed:
receive a sleep level query generated by a gesture input on a touch interface of the earphone; and
wherein the sleep sufficiency subroutine comprising computer readable instructions that when generated:
display a clock on the display,
generate a second graphical representation of the proportion of the actual sleep duration value relative to the target sleep duration value comprising a third graphical element representing the proportion with a color, the second graphical representation to decrease the cognitive load of the user in interpreting the sufficiency of the sleep,
generate a sound representation of the proportion of the actual sleep duration value relative to the target sleep duration value comprising at least one of (i) a tone representing the proportion and (ii) a word describing the proportion, the sound representation played on the speaker of the earphone,
wherein at least one of the physiological data and the indicator data received from the earphone at a rate of between 20 Hz and 700 Hz, and
wherein the coordination hub is pairable with a device generating the communication notification is recognizable by the device as an earphone device through a pairing communication protocol.
